(19) United States
(12) Patent Application Publication
(10) Pub. No.: US 2025/0299780 A1
(43) Pub. Date: Sep. 25, 2025

(54) SYSTEM AND METHODS FOR PREDICTING FEATURES OF BIOLOGICAL SEQUENCES

(71) Applicant: Dyno Therapeutics, Inc., Watertown, MA (US)

(72) Inventors: Lauren Wheelock, Cambridge, MA (US); Sam Sinai, Watertown, MA (US); Jeffrey Gerold, Watertown, MA (US)

(21) Appl. No.: 18/863,560

(22) PCT Filed: May 5, 2023
(86) PCT No.: PCT/US2023/066686

§ 371 Date: Nov. 6, 2024

Related U.S. Application Data
(60) Provisional application No. 63/339,224, filed on May 6, 2022.
(60) Provisional application No. 63/343,881, filed on May 19, 2022.
(60) Provisional application No. 63/412,169, filed on Sep. 30, 2022.
(60) Provisional application No. 63/426,238, filed on Nov. 17, 2022.

(51) Int. Cl. G16B 40/20; G06N 20/00; G16B 5/20; G16B 30/00

(52) U.S. Cl. CPC G16B 40/20; G06N 20/00; G16B 5/20; G16B 30/00

(57) ABSTRACT

Techniques for predicting performance of biological sequences. The technique may include using a statistical model configured to generate output indicating predictions for an attribute of biological sequences, the biological sequences generated using a machine learning model trained on training data. The statistical model is configured to allow for at least some of the predictions to occur outside a distribution of labels in the training data.

### CROSS-REFERENCE TO RELATED APPLICATIONS

This application claims priority to U.S. Provisional Application No. 63/339,224, filed May 6, 2022, U.S. Provisional Application No. 63/343,881, filed May 19, 2022, U.S. Provisional Application No. 63/412,169, filed Sep. 30, 2022, and U.S. Provisional Application No. 63/426,238, filed Nov. 17, 2022, each of which is hereby incorporated by reference in its entirety.

### FIELD

Aspects of the technology described herein relate to using statistical models for predicting performance of biological sequences, including those generated using a machine learning model.

### BACKGROUND

Advances in engineering biomolecules, such as proteins, have allowed for the implementation of novel biological molecules in many areas of biotechnology and medicine. These new biological molecules may have improved characteristics in comparison to their wildtype versions.

### SUMMARY

Some embodiments are directed to a method for predicting performance of biological sequences, comprising: using at least one computer hardware processor to perform: accessing a plurality of biological sequences and model scores associated with the plurality of biological sequences, wherein the plurality of biological sequences and model scores are generated using a machine learning model trained on training data comprising biological sequences and labels for an attribute of the biological sequences; accessing a statistical model configured to generate output indicating predictions for the attribute of the plurality of biological sequence, wherein the statistical model is configured to allow for at least some of the predictions to occur outside a distribution of labels in the training data; and generating, using the statistical model, the plurality of biological sequences, and the model scores, an output indicating a predicted distribution of labels for the attribute of the plurality of biological sequences.

In some embodiments, the statistical model allows for at least some of the predictions to occur outside a range of the distribution of labels in the training data. In some embodiments, the statistical model is configured to allow for at least some of the predictions to occur outside a distribution of models scores generated by ensembling the model scores.

In some embodiments, the method further comprises determining, using the output indicating the predicted distribution of labels, a likelihood of the plurality of biological sequences comprising at least one biological sequence having a measurement for the attribute greater than the labels. In some embodiments, the method further comprises determining, using the output indicating the predicted distribution of labels, a number of biological sequences from among the plurality of biological sequences as having a value for the attribute above a threshold value.

In some embodiments, the plurality of biological sequences is a first plurality of biological sequences, the method further comprising generating, based on the output indicating the predicted distribution of labels, a second plurality of biological sequences at least in part by using the machine learning model to obtain as output the second plurality of biological sequences. In some embodiments, the predicted distribution of labels of the attribute comprises a distribution of values corresponding to predictions of the attribute for the plurality of biological sequences.

In some embodiments, the method further comprises manufacturing at least some of the plurality of biological sequences. In some embodiments, the method further comprises: selecting, based on the predicted distribution of labels for the attribute, a subset of the plurality of biological sequences; and manufacturing the subset of the plurality of biological sequences.

In some embodiments, the plurality of biological sequences is a first plurality of biological sequences, the model scores is a first set of model scores, and the output is a first output, and wherein the method further comprises: accessing a second plurality of biological sequences and a second set of model scores associated with the second plurality of biological sequences; generating, using the statistical model, the second plurality of biological sequences, and the second set of model scores, a second output indicating a predicted distribution of labels for the attribute for the second plurality of biological sequences; and selecting the first plurality of biological sequences or the second plurality of biological sequences based on the first output and the second output.

In some embodiments, the method further comprises: manufacturing, based on the selecting, the first plurality of biological sequences or the second plurality of biological sequences.

In some embodiments, the model scores include at least one model score associated with each of the plurality of biological sequences. In some embodiments, the machine learning model includes a regression model, and the model scores include regression estimates associated with the plurality of biological sequences.

In some embodiments, generating the output using the statistical model, the plurality of biological sequences, and the model scores further comprises identifying, using the model scores, an estimate for at least one parameter of a probability distribution for the plurality of biological sequences. In some embodiments, generating the output using the statistical model, the plurality of biological sequences, and the model scores further comprises determining, for each of the plurality of biological sequences, a probability distribution. In some embodiments, determining the probability distribution for each of the plurality of biological sequences further comprises identifying estimates for parameters of the probability distribution for each of the plurality of biological sequences based on the model scores. In some embodiments, identifying parameters of the probability distribution for each of the plurality of biological sequences further comprises identifying means and variances for the model scores, each mean and each variance corresponding to one biological sequence of the plurality of biological sequences. In some embodiments, determining the probability for each of the plurality of biological sequences further comprises determining a posterior distribution for each of the plurality of biological sequences and identifying estimates for parameters of the posterior distribution for each of the plurality of biological sequences based on the model scores.

In some embodiments, the statistical model comprises a multimodal model having a first mode and a second mode, and identifying estimates for parameters of the probability distribution for each of the plurality of biological sequences further comprises identifying a first set estimates for parameters associated with the first mode and a second set of estimates for parameters associated with the second mode. In some embodiments, the statistical model includes at least one Gaussian mixture model comprising the first mode and the second mode.

In some embodiments, the statistical model includes a first regression model trained on biological sequences and labels associated with the first mode and a second regression model trained on biological sequences and labels associated with the second mode, and wherein identifying estimates for parameters of the probability distribution further comprises using the first regression model to identify the first set of estimates for parameters associated with the first mode and using the second regression model to identify the second set of estimates for parameters associated with the second mode.

In some embodiments, generating the output indicating the predicted distribution of labels for the attribute of the plurality of biological sequences further comprises using the first set of estimates for parameters associated with the first mode to generate a predicted distribution of labels associated with the first mode and using the second set of estimates for parameters associated with the second mode to generate a predicted distribution of labels associated with the second mode.

In some embodiments, the statistical model includes a parameter relating to a sequence distance metric, and generating the output indicating the predicted distribution of labels further comprises using an estimate for the parameter relating to a sequence distance metric to adjust the predictions generated by the statistical model.

In some embodiments, the plurality of biological sequences comprises polypeptide sequences. In some embodiments, the plurality of biological sequences comprises sequences for dependoparvovirus capsid proteins. In some embodiments, the plurality of biological sequences comprises variants of a wildtype dependoparvovirus capsid protein. In some embodiments, the attribute is transduction efficiency for a target tissue type, and the labels comprise values of transduction efficiency for dependoparvovirus capsid proteins. In some embodiments, the attribute includes packaging efficiency, and the labels comprise values of packaging efficiency for the dependoparvovirus capsid proteins.

Some embodiments are directed to a system comprising: at least one hardware processor; and at least one non-transitory computer-readable storage medium storing processor-executable instructions that, when executed by the at least one hardware processor, cause the at least one hardware processor to perform the method comprising: accessing a plurality of biological sequences and model scores associated with the plurality of biological sequences, wherein the plurality of biological sequences and model scores are generated using a machine learning model trained on training data comprising biological sequences and labels for an attribute of the biological sequences; accessing a statistical model configured to generate output indicating predictions for the attribute of the plurality of biological sequence, wherein the statistical model is configured to allow for at least some of the predictions to occur outside a distribution of labels in the training data; and generating, using the statistical model, the plurality of biological sequences, and the model scores, an output indicating a predicted distribution of labels for the attribute of the plurality of biological sequences.

Some embodiments are directed to at least one non-transitory computer-readable storage medium storing processor-executable instructions that, when executed by at least one hardware processor, cause the at least one hardware processor to perform the method comprising: accessing a plurality of biological sequences and model scores associated with the plurality of biological sequences, wherein the plurality of biological sequences and model scores are generated using a machine learning model trained on training data comprising biological sequences and labels for an attribute of the biological sequences; accessing a statistical model configured to generate output indicating predictions for the attribute of the plurality of biological sequence, wherein the statistical model is configured to allow for at least some of the predictions to occur outside a distribution of labels in the training data; and generating, using the statistical model, the plurality of biological sequences, and the model scores, an output indicating a predicted distribution of labels for the attribute of the plurality of biological sequences.

Some embodiments are directed to a method, comprising: accessing a first plurality of biological sequences and a first set of model scores associated with the first plurality of biological sequences; accessing a statistical model configured to generate output indicating estimates for at least one feature of a biological sequence; and generating, using the statistical model, the first plurality of biological sequences, and the first set of model scores, a first output indicating estimates of the at least one feature for the first plurality of biological sequences.

In some embodiments, the first output includes a distribution of values corresponding to the estimates of the at least one feature for the first plurality of biological sequences.

In some embodiments, the method further comprises selecting, based on the estimates of the at least one feature, a subset of the first plurality of biological sequences; and manufacturing the subset of the first plurality of biological sequences.

In some embodiments, the method further comprises: accessing a second plurality of biological sequences and a second set of model scores associated with the second plurality of biological sequences; generating, using the statistical model, the second plurality of biological sequences, and the second set of model scores, a second output indicating estimates of the at least one feature for the second plurality of biological sequences; and selecting the first plurality of biological sequences or the second plurality of biological sequences based on the first output and the second output. In some embodiments, the method further comprises manufacturing, based on the selecting, the first plurality of biological sequences or the second plurality of biological sequences.

In some embodiments, the first set of model scores include regression estimates associated with the first plurality of biological sequences. In some embodiments, the first set of model scores include model scores associated with each of the first plurality of biological sequences. In some embodiments, generating the first output further comprises identifying means and variances for the first set of model scores, each mean and each variance corresponding to the model scores associated with one of the first plurality of biological sequences.

In some embodiments, the statistical model includes at least one Gaussian mixture model. In some embodiments, generating the first output further comprises: sampling, using the at least one Gaussian mixture model, distributions of a first feature of the at least one feature for the first plurality of biological sequences; and identifying estimates of the first feature based on the distributions. In some embodiments, the sampling further comprises: sampling, using the at least one Gaussian mixture model, a distribution of the first feature for each of the first plurality of biological sequences.

In some embodiments, the statistical model was trained using training data that includes a second set of model scores associated with a second plurality of biological sequences and measurement data for the second plurality of biological sequences. In some embodiments, at least some of the estimates have values greater than values of the measurement data. In some embodiments, at least some of the estimates have values greater than a highest value of the measurement data. In some embodiments, at least some of the first plurality of biological sequences having a model score greater than a threshold value is estimated to have a value for the at least one feature greater than for the second plurality of biological sequences.

In some embodiments, the method further comprises training the statistical model, the training comprising identifying at least one parameter of the statistical model using the second set of model scores and the measurement data for the second plurality of biological sequences. In some embodiments, identifying the at least one parameter further comprises: identifying means and variances for the second set of model scores; identifying means and variances for the measurement data; and identifying the at least one parameter based on the means and variances for the second set of model scores and the means and variances for the measurement data. In some embodiments, identifying the at least one parameter further comprises using at least one isotonic regression model to identify the at least one parameter.

In some embodiments, at least one parameter of the statistical model relates a calibration value for estimates of the at least one feature based on edit distance of a biological sequence to a wildtype biological sequence.

In some embodiments, the first plurality of biological sequences comprises protein sequences. In some embodiments, the first plurality of biological sequences comprises sequences for dependoparvovirus capsid proteins. In some embodiments, the dependoparvovirus is an adeno-associated dependoparvovirus (AAV). In some embodiments, the first plurality of biological sequences comprises variants of a wildtype dependoparvovirus capsid protein. In some embodiments, the at least one feature includes transduction efficiency for a target tissue type, and the estimates include values of transduction efficiency for dependoparvovirus capsid proteins. In some embodiments, the at least one feature includes packaging efficiency, and the estimates include values of packaging efficiency for the dependoparvovirus capsid proteins.

Some embodiments are directed to a system comprising: at least one hardware processor; and at least one non-transitory computer-readable storage medium storing processor-executable instructions that, when executed by the at least one hardware processor, cause the at least one hardware processor to perform a method. The method comprises accessing a first plurality of biological sequences and a first set of model scores associated with the first plurality of biological sequences; accessing a statistical model configured to generate output indicating estimates for at least one feature of a biological sequence; and generating, using the statistical model, the first plurality of biological sequences, and the first set of model scores, a first output indicating estimates of the at least one feature for the first plurality of biological sequences.

Some embodiments are directed to at least one non-transitory computer-readable storage medium storing processor-executable instructions that, when executed by at least one hardware processor, cause the at least one hardware processor to perform: accessing a first plurality of biological sequences and a first set of model scores associated with the first plurality of biological sequences; accessing a statistical model configured to generate output indicating estimates for at least one feature of a biological sequence; and generating, using the statistical model, the first plurality of biological sequences, and the first set of model scores, a first output indicating estimates of the at least one feature for the first plurality of biological sequences.

### BRIEF DESCRIPTION OF DRAWINGS

Various aspects and embodiments will be described with reference to the following figures. The figures are not necessarily drawn to scale.

FIG. 1 is a diagram of an illustrative process for predicting performance of biological sequences using a statistical model configured to allow for at least some predictions to occur outside the label distribution of training data used in training the machine learning model that generated the biological sequences.

FIG. 2 is a schematic for three illustrative distributions: (1) a distribution of labels in the training data, (2) a distribution of model scores, and (3) a distribution of measurements obtained for designed biological sequences.

FIG. 3 is a schematic for two illustrative distributions: (1) a distribution of labels in the training data, and (2) a distribution of model scores.

FIG. 4 is a schematic of two illustrative distributions: (1) a distribution of labels in the training data, and (3) predicted distribution of labels.

FIG. 5 is a flow chart of an illustrative process for predicting features of machine-guided designed biological sequences, in accordance with some embodiments of the technology described herein.

FIG. 6 is a flow chart of an illustrative process for predicting features of machine-guided designed biological sequences, in accordance with some embodiments of the technology described herein.

FIG. 7 is a block diagram of an illustrative computer system that may be used in implementing some embodiments of the technology described herein.

FIGS. 8A-8H are plots showing comparison between forecast and point predictions in describing the right tail statistics of designed libraries. a) Distance from true (right-tail) distribution as measured by KS two-sample score for ensemble point estimate and our forecast on two RNA landscapes and five distinct designs per landscape (10 total forecasts). b) Top centile confidence interval coverage for RNA landscape 1 (14 nt) and RNA landscape 2 (50 nt). c, e, g) Distance from true distribution fit for ensemble point and forecast. d, f, h) Confidence interval coverage based on the number of samples above a certain measured performance. c, d) For the AAV capsid design problem. e, f) For the GB1 binding landscape. g, h) For the GFR florescence landscape.

FIG. 9 is a plot showing the ensemble, forecast, and measured values for the 99^{th} percentile, mean of the top percentile, and maximum value for AAV, GFP, and GB1 experiments, normalized to the maximum ground-truth measured value for each experiment.

FIG. 10 is a plot showing the ensemble, forecast, and true values for the 99^{th} percentile, mean of the top percentile, and maximum value for the RNA experiments.

### DETAILED DESCRIPTION

Machine learning-guided approaches for designing biological sequences, such as nucleic acids and proteins, have the potential to aid in discovery of non-naturally occurring biological molecules that provide value in many areas of biotechnology, medicine, and healthcare. These biological molecules may have one or more enhanced features in comparison to other similar types of biological molecules (e.g., wildtype), which may allow for improved drugs and therapeutics.

In the context of using machine learning-guided approaches in designing viral vectors for delivering various payloads to cells, such as dependoparvoviruses (e.g. adeno-associated dependoparvoviruses, e.g. adeno-associated viruses (AAVs)), enhanced features of an improved dependoparvovirus capsid protein may include one or more of increased transduction to a particular tissue type (e.g. eye, brain, liver, skeletal muscle, cardiac muscle), decreased transduction to a particular off-target tissue (e.g., liver), and increased production efficiency, for example. Other features of an improved dependoparvovirus capsid protein may include its alterations (e.g., amino acid substitutions, deletions, insertions) in comparison to another dependoparvovirus capsid protein (e.g. a wildtype AAV) and its edit distance to another dependoparvovirus capsid protein.

The inventors have recognized the potential in using machine learning-guided approaches in designing biological sequences, particularly in increasing the speed in identifying new biological sequences with desired enhanced properties. The inventors have also recognized that certain limitations may exist on a research and development pipeline that implements a machine-guided design approach because these approaches can generate a large number of biological sequences that then need to be experimentally evaluated and the challenges with allocating time and resources, particularly when the number of biological sequences may exceed experimental capacity. For example, a library of biological sequences generated using a machine-guided design approach may include up to 10⁵ sequences for a high-throughput experiment, and the design process may take a few weeks to complete. In contrast, the process between the sequence design stage and experimental or clinical validation of those sequences may be resource intensive, both in terms of cost and time, particularly in comparison to the resources devoted to design of the original sequence library. For instance, a high-throughput validation experiment for a library of 10⁵ sequences may take on the order of many months to a year to complete from production of the sequence library, completion of animal studies, processing to tissues, and analyzing the data from these studies.

Another challenge that arises from a research and development pipeline that typically involves a long experimental and validation timeline for a given sequence library is a delay of feedback on the machine-learning guided approach used to design the library because any experimental data used to evaluate performance of the machine-learning approach would be obtained after completion of the validation process. This delay in feedback may deter improvements and iterations on the machine-learning design approach, which may impact the design of successive libraries of biological sequences. For example, in situations where a series of libraries are being successively designed using machine-learning approaches, feedback from previous iterations may not be included in updates and improvements to the design process before the next iteration of sequence design because experimental data from validation studies have not yet been obtained and analyzed.

To address some of the aforementioned challenges, the inventors have developed computational techniques for predicting performance of biological sequences generated using machine-guided design. These computational techniques may be referred to herein as “forecasting model(s)” and can be implemented in a research and development pipeline to predict the likelihood of biological sequences having one or more features. As used herein, a “feature” of a biological sequence may correspond to the biological sequence having a particular value for an attribute. In turn, these predictions can be used to inform decision-making during the validation process for those sequences, including assisting with decisions related to allocation resources in the research and development pipeline. In instances where resources are limited and unable to accommodate all of the designed biological sequences, these computational techniques for predicting features of biological sequences may be particularly beneficial and used to select which biological sequences to include in validation experiments. For example, some embodiments may involve using these computational techniques to generate predictions for multiple libraries of biological sequences, and, depending on those predictions, the libraries may be prioritized for validation such that the library predicted as having the most desired feature(s) has priority over a library predicted as having a lower likelihood of having the desired feature(s).

During biological sequence design, model scores may be generated depending on the machine-learning guided approach used. As an example, if regression models are implemented to design biological sequences, the regression models may output model scores associated with the biological sequences. When multiple regression models are implemented to design biological sequence, then the output from the models may include a model score for each of the regression models for a single biological sequence. As a result, model scores associated with a library of biological sequences may include a set of model scores for each biological sequence in the library. According to the computational techniques described herein, a statistical model may be used to relate the model scores generated for the biological sequences to predictions for feature(s) of the biological sequences. Implementing the statistical model to predict feature(s) of the biological sequences may involve using the model scores as an input to the statistical model to obtain estimates of the feature(s).

The inventors have recognized that some of the challenges in using machine-guided approaches in designing biological molecules arise because of limited existing training data. When the desired goal of using machine-guided approaches is to design biological sequences with improved features, such as enhanced properties or a desired amount of variation in comparison to a wildtype version, it can be challenging to obtain biological sequences having feature(s) at a level occurring outside the training data. For instance, the aim with machine-guided design may be to obtain biological sequences having model scores that are higher than what is observed in the training data. This concept may be considered as “distribution shift” in that the sequences being designed shift away from the performance distribution of the training data. Thus, one challenge in developing computational techniques for predicting feature(s) of biological sequences designed with machine-guided approaches is making high-confident predictions for features that to occur outside the distributions of the training data. Accordingly, the computational techniques developed by the inventors and described herein allow for such a distribution shift when making predictions by identifying parameter(s) of a statistical model using the model scores. The parameter(s) of the statistical model are calibrated to the training data and allow for performance of feature(s) of biological sequences to differ from or exceed the performance of the training data, thus accounting for distribution shift. In particular, the inventors have developed a “semi-calibration” technique that transitions from calibrated predictions at or near the center of the distribution from the training data towards an uncalibrated, out of distribution predictions towards the limits of the distribution from the training data.

In some instances, the inventors have recognized that feature performance of biological sequences designed using machine-guided approaches have a multi-modal distribution. According to some embodiments, the statistical model is a multimodal model (e.g., Gaussian mixture model). In such embodiments, estimates for parameter(s) multimodal model may be identified using model scores for the designed biological sequences. In some embodiments, model scores are transformed into estimates for parameters of the multimodal model for each sequence and predictions for a library of biological sequence are obtained by sampling each of those models.

The inventors have further recognized that the model scores may be subject to bias depending on the edit distance of a designed biological sequence to its wildtype version. Accordingly, the inventors have developed a correction technique that involves implementing a bias-correcting parameter that varies depending on a biological sequence's edit distance to a wildtype sequence.

It should be appreciated that the various aspects and embodiments described herein be used individually, all together, or in any combination of two or more, as the technology described herein is not limited in this respect.

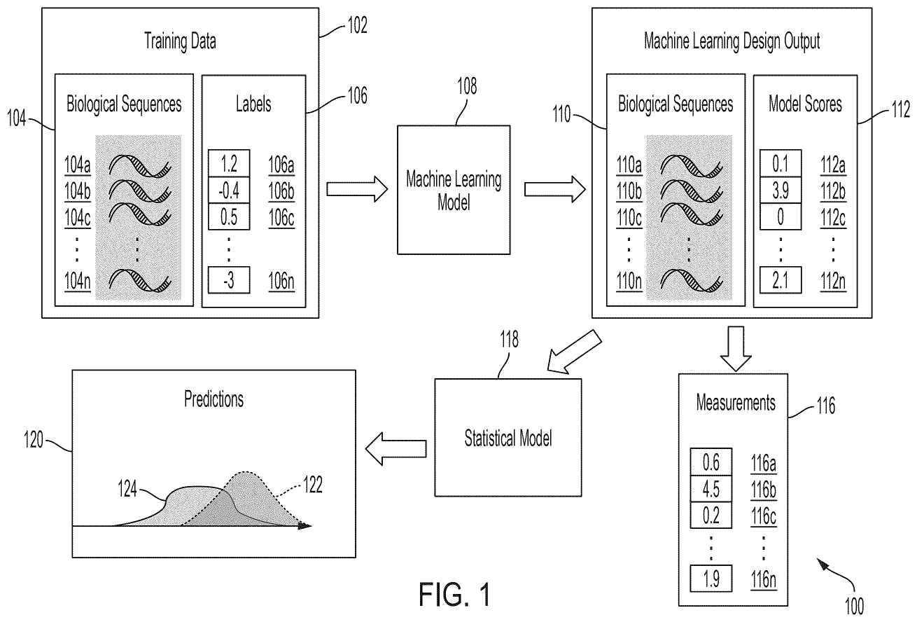

FIG. 1 is a diagram of an illustrative process 100 for predicting performance of biological sequences generated using a machine learning model, which may include accessing the biological sequences and model scores associated with the biological sequences, accessing a statistical model configured to generate output indicating predictions for an attribute of the biological sequences, and using the statistical model, the biological sequences, and the model scores, to generate an output indicating a predicted distribution of labels for the attribute of the biological sequences.

As shown in FIG. 1, biological sequences 110 and model scores 112 associated with biological sequences 110 are generated using machine learning model 108. Machine learning model 108 has been trained on training data 102, which includes biological sequences 104 and labels 106 for an attribute of biological sequences 104. Process 100 includes statistical model 118 configured to generate output indicating predictions 120 for the attribute for biological sequences 110 generated using machine learning model 108. Labels 106 of training data 102 form a label distribution for the attribute of biological sequences 104, which may be referred to herein as a “training data label distribution.” FIG. 1 shows a schematic of training data label distribution 124 for illustrative purposes. According to the techniques described herein, statistical model 118 is configured to allow for at least some of predictions 120 to occur outside the training data label distribution. Process 100 involves using statistical model 118, biological sequences 110, and model scores 112 to generate an output indicating a predicted distribution of labels for the attribute of biological sequences 110. FIG. 1 shows a schematical of predicted distribution of labels 122. As shown in FIG. 1, the predicted distribution of labels 122 is shifted to the right relative to the training data label distributions, indicating that some of the predictions generated by statistical model occur outside the training data label distribution.

In some embodiments, process 100 includes obtaining measurements 116 for biological sequences 110 generated using machine learning model 108. In some embodiments, the predicted label distribution 112 for biological sequences 110 is used to evaluate whether to proceed with a study (e.g., an in vivo animal study, such as a mouse study, a non-human primate study) to obtain measurements 116. In this way, the predictions 120, including predicted distribution of labels 122, generated by statistical model 118 may be used to inform experimental study decisions. As predictions 120 are indicative of performance of biological sequences 110 in a potential future experimental study if conducted, statistical model 118 may be considered to “forecast” performance of biological sequences designed using machine learning approaches.

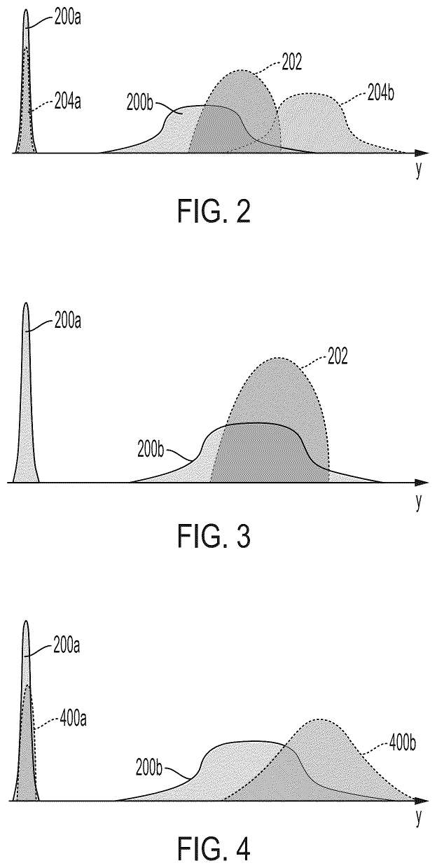

Conventional techniques for predicting performance of biological sequences designed using a machine learning model involve naively using model scores as an estimate of performance, such as by ensembling the model scores generated by the machine learning model. In the context of FIG. 1, such an approach involves obtaining an ensemble of model scores 112 associated with biological sequences 110. For instance, if machine learning model 108 used to generate biological sequences 110 generates point estimates as model scores 112, the conventional approach dictates the ensembled point estimates as the predicted performance of biological sequences 110. Further explanation of this conventional approach is described in Section A. This approach has several disadvantages and tends to significantly underestimate the actual performance of the designed biological sequences. Within a context where labels 106, model scores 112, and measurements 116 all correspond to the same attribute, FIG. 2 is a schematic for three illustrative distributions: (1) a distribution of labels 200 in the training data, (2) a distribution of model scores 202, (3) a distribution of measurements 204 obtained for designed biological sequences. In FIG. 2, the range of values in each distribution is shown along the y-axis and the shape of the distribution curve indicates the relative probability for those values.

FIG. 2 illustrates some of the disadvantages with the conventional approach of using ensembled model scores to predict performance of biological sequences. First, one disadvantage is the distribution of model scores 202 is unimodal whereas the distribution of measurements 204 obtained for designed biological sequences is multimodal. As shown in FIG. 2, the distribution of labels 200 in the training data is multimodal because the labels correspond to measurements obtained for the biological sequences in the training data. These multimodal distributions may arise because at several points in the experimental pipeline biological sequences may “drop out” failing to produce enough signal of the attribute to measure or reliably approximate a label (e.g., due to failure of a protein to fold). As a result, the distribution of measurements 204 may include one mode 204a at lower performance relative to another mode 204b. In this way, mode 204a of the distribution of measurements may correspond to “broken” biological sequences and mode 204b may correspond to “functional” biological sequences. In addition, FIG. 2 shows the distribution of labels includes mode 200a for “broken” biological sequences included in the training data and mode 200b for “functional” biological sequences included in the training data. To address this disadvantage, statistical model 118 may include a Gaussian mixture model (GMM), e.g., a bimodal GMM, and using a statistical inference technique to distinguish between “functional” and “broken” biological sequences.

Second, another disadvantage is that the conventional approach with using model scores as a proxy for performance of biological sequences is that there is unaccounted for distribution (covariate) shift and no label shift. One of the objectives in designing biological sequences is to produce sequences that outperform the best biological sequences in the training data, this may result in distribution (covariate) shift because the biological sequences being designed are within untested areas of sequence space and label shift because the anticipated measurements for the designed biological sequences will outperform the labels of the training data. As shown in FIG. 2, the distribution of model scores 202 falls within the same range of the “functional” mode of the distribution of labels 200b in the training data, but does not account for the label shift (along the y-axis) of the “functional” mode 204b of the measurements from the “functional” mode 200b of the labels in the training data. The techniques described herein address this disadvantage by using a statistical model that allows for at least some of predictions for performance of the designed biological sequences to occur outside the range of the distribution of labels in the training data, thus allowing for some label shift.

A third disadvantage of using model scores naively to predict performance of biological sequences is that this approach tends to underestimate the frequency events that are often more rare at the sequence level, such as the occurrence of high valued biological sequences, including biological sequences having high performance relative to others. In FIG. 2 these “rare” events are illustrated by the elongated tails of the distribution of measurements in the “functional” mode 204b (as well as the “functional” mode 200b of the distribution of labels). In particular, the high performing biological sequences in mode 204b of the distribution of measurements illustrated by the elongated right tail along the y-axis. To address this disadvantage, the techniques described herein may involve statistical approaches that allow for more accurate prediction of frequency of rare events, e.g., high performing biological sequences.

FIG. 3 and FIG. 4 illustrate how improved techniques for predicting performance of biological sequences as described herein generate a more accurate representation of how the biological sequences perform. FIG. 3 shows a schematic for two illustrative distributions: (1) a distribution of labels 200 in the training data, and (2) a distribution of model scores 202. FIG. 4 shows a schematic with the same distribution of labels 200 in the training data and predicted distribution of labels 400 generated using the techniques described herein (e.g., using statistical model 118). As shown in FIG. 4, these improved techniques result in a predicted distribution of labels for designed biological sequences having two modes: mode 400a corresponding to “broken” biological sequences that may occur as a result of design and mode 400b corresponding to “functional” biological sequences. For mode 400b, the predicted distribution of labels occurs outside the mode 200b of the distribution of labels, where the predicted distribution of labels for mode 400b occur outside the range of the distribution of labels for mode 200b. As shown in FIG. 4, this is represented by mode 400b being shifted relative to mode 200b along the y-axis, indicating that some of the predictions associated with mode 400b have a higher value for the attribute of interest than the labels associated with mode 200b. In addition, mode 400b has elongated tails compared to distribution of model scores 202 shown in FIG. 3. This illustrates how the techniques described herein may allow for more accurate prediction of frequency of rare events, including predicting of high performing biological sequences (as represented by a the right tail of along the y-axis of mode 400b).

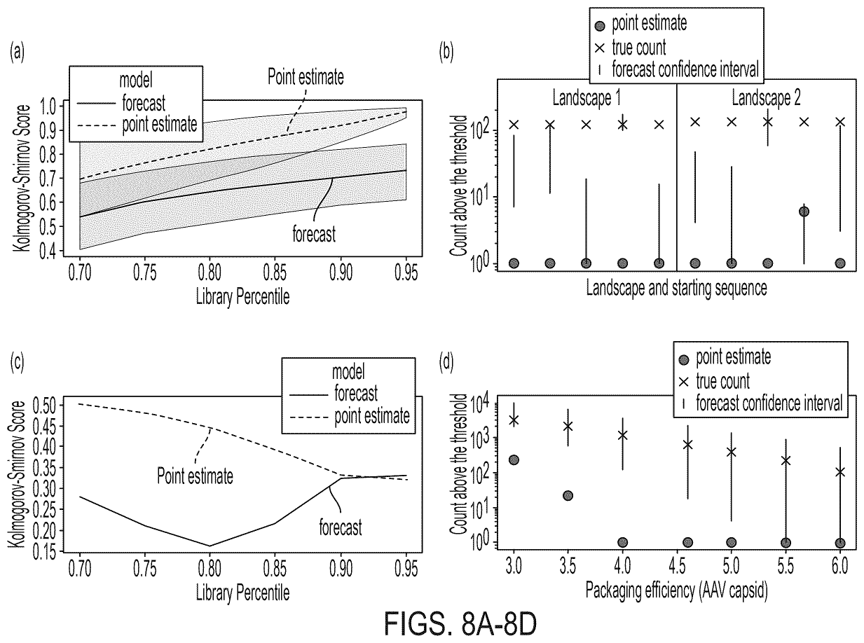

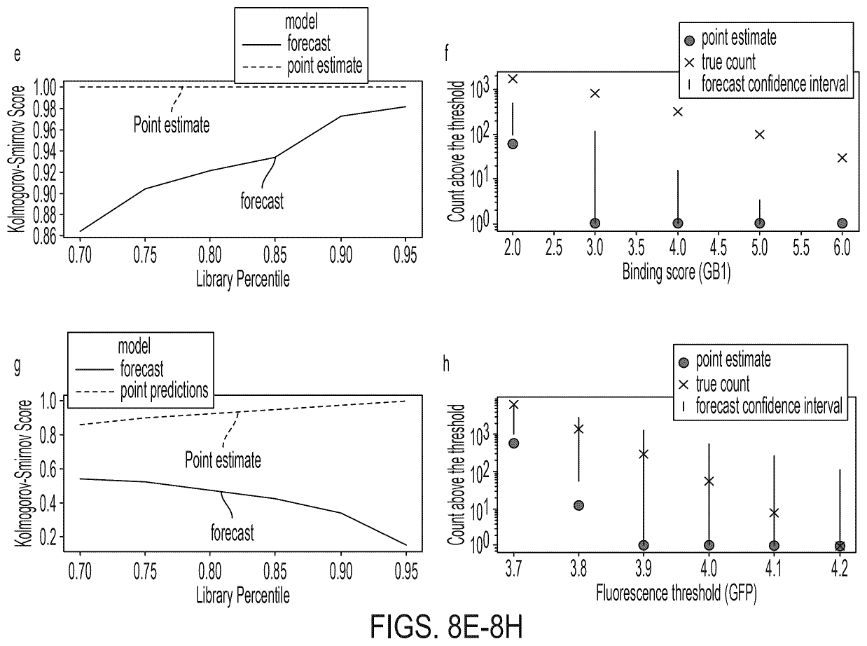

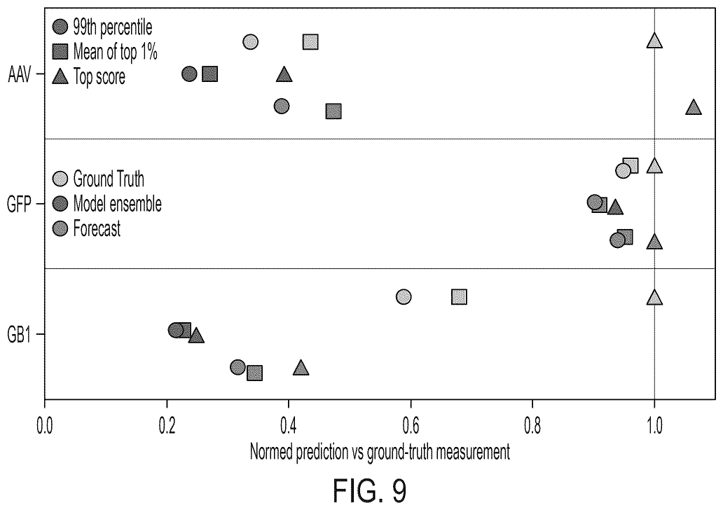

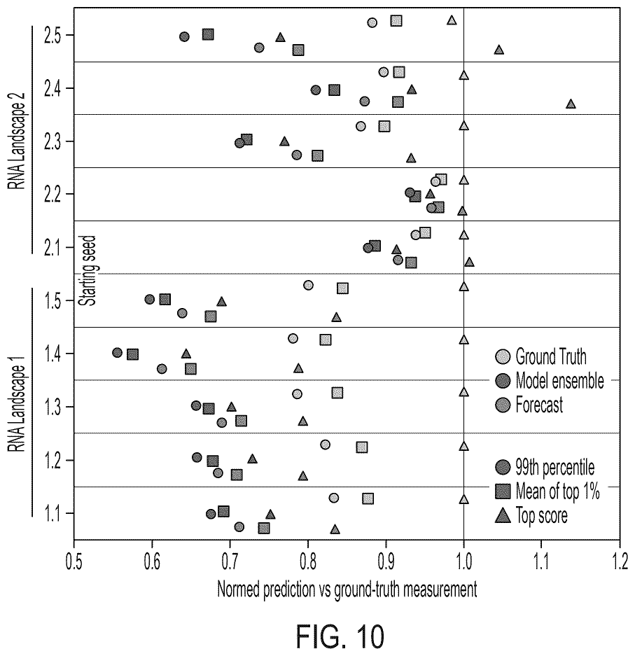

The benefits of the techniques are shown in FIGS. 8A-8H, FIG. 9, and FIG. 10, which are described in more detail in Section A. In particular, these results illustrate how the “forecasting” techniques described herein in general outperform predictions of performance based on ensembled model scores (e.g., ensembled point estimates), particularly in comparison to the measurements for the biological sequences (here, the “ground truth”). For example, FIG. 9 illustrates how in three different protein design contexts (AV, GFP, and GB1) the techniques described herein (labeled as “forecast”) provide more accurate predictions because they are closer in value to the ground truth than ensembled point estimates (labeled as “model estimates”). FIGS. 8B, 8D, 8F, 8H, and FIG. 10 show similar results and are described further in Section A.

Returning to FIG. 1, in some embodiments, training data 102 may include one or more labels 106 associated with individual biological sequences 104. As shown in FIG. 1, label 106a is associated with biological sequence 104a, label 106b is associated with biological sequence 104b, label 106c is associated with biological sequence 106c, . . . label 106n is associated with biological sequence 104n. Although only a single label associated with an individual biological sequence is shown in FIG. 1, it should be appreciated that multiple labels may be associated with a particular biological sequence to form training data 102, as aspects of the present application are not limited to the number of labels associated with an individual biological sequence of training data 102 used to train machine learning model 108. Labels 106 may correspond to measurements experimentally obtained for biological sequences 104. In the sequence design contexi, such experimentally obtained measurements for designed biological sequences may be referred to as “fitness measurements.” As shown in FIG. 1, labels 106 may include continuous-valued labels. The continuous values may be normalized, unnormalized, or have any suitable range of values, as aspects of the present application are not limited to the particular range of values used as labels 106 of training data 102.

Training data 102 may include a number of biological sequences, e.g., between approximately 100 to approximately 10,000,000. In some embodiments, training data 102 may include a number of biological sequences in a range of 100 to 100,000, 100 to 200,000, 100 to 500,000, 100 to 1,000,000, 100 to 10,000,000, 100,000 to 10,000,000, or any value or range of values in the range of 100 to 10,000,000. Biological sequences 104 of training data 102 may include biological sequences generated by making one or more mutations (e.g., substitutions, deletions, insertions) from a starting sequence (e.g., wild-type sequence). In some embodiments, individual biological sequences 104 may have between 1 and 3 mutations, between 1 and 5 mutations, between 1 and 10 mutations, between 1 and 20 mutations, between 1 and 50 mutations. In some embodiments, biological sequences 104 include nucleic acid sequences (e.g., deoxyribonucleic acid (DNA), ribonucleic acid (RNA), and different types of RNA, such as messenger RNA (mRNA)). In some embodiments, biological sequences 104 include amino acid sequences (e.g., a polypeptide sequence, a region of a polypeptide sequence for a protein). Biological sequences 104 may have any suitable length (e.g., number of nucleotides for a nucleic acid sequence, number of amino acids for a polypeptide sequence). In some embodiments, biological sequences 104 have a one-hot ended sequence format used to train machine learning model 108. Sections A.4.1.1, A.4.1.2, A.4.1.3, and A.4.1.4 describe examples of training data sets.

As shown in FIG. 1, some embodiments may involve training machine learning model 108 using training data 102. Training machine learning model 108 may involve training machine learning model 108 such that machine learning model 108 is configured to generate an output indicating biological sequence(s) and model score(s) associated with the biological sequence(s), such as biological sequences 110 and model scores 112 shown in FIG. 1. In some embodiments, machine learning model 108 includes multiple models (e.g., multiple regression models) used to generate biological sequences 110 and associated model scores 112. In some embodiments, each of the multiple models may be individually trained using training data 102. In some embodiments, two or more of the multiple models may be jointly trained using training data 102. For example, training data 102 may include a set of labels for a first attribute and a set of labels for a second attribute associated with biological sequences 104, and two models jointly trained on such training data may balance optimization of the first and second attributes in generating designed biological sequences.

Machine learning model 108 have any architecture suitable for generating biological sequences. In some embodiments, machine learning model 108 includes a convolutional neural network. In some embodiments, machine learning model 108 includes a recurrent neural network. In some embodiments, machine learning model 108 includes a multi-layer perception. In some embodiments, machine learning model 108 includes a random forest model. In some embodiments, machine learning model 108 includes a regression model. Examples of regression models include linear regression models (e.g., simple linear regression, multiple linear regression), polynomial regression models, and Bayesian linear regression models. In some embodiments, the regression model has a convolutional neural network architecture. An example of an algorithm that implements machine learning model(s) suitable for designing biological sequences and may be used to generate biological sequences 110 and model scores 112 is CMA-ES, which is described in N. Hansen and A. Ostermeier, “Completely derandomized self-adaption in evolution strategies,” Evolutionary computation, 9(1):159-195, 2001, which is incorporated herein by reference in its entirety. Another example of an algorithm that implements machine learning model(s) suitable for designing biological sequences and may be used to generate biological sequences 110 and model scores 112 is CbAS, which is described in D. Brookes, H. Park, and J. Listgarten, “Conditioning by adaptive sampling for robust design,” arXiv preprint arXiv:1901.10060, 2019, which is incorporated herein by reference in its entirety. Another example of an algorithm that implements machine learning model(s) suitable for designing biological sequences and may be used to generate biological sequences 110 and model scores 112 is Adalead, which is described in S. Sinai, R. Wang, A. Whatley, S. Slocum, E. Locane, and E. Kelsic, “Adalead: A simple and robust adaptive greedy search algorithm for sequence design,” arXiv preprint arXiv:2010.02141, 2020, which is incorporated herein by reference in its entirety.

In some embodiments, machine learning model 108 is used to generate biological sequences 110 and model scores 112, where one or more model scores 112 is associated with a particular biological sequence 110. As shown in FIG. 1, model score 112a is associated with biological sequence 110a, model score 112b is associated with biological sequence 110b, model score 112c is associated with biological sequence 110c, . . . model score 112n is associated with biological sequence 110n. Although only a single model score associated with an individual biological sequence is shown in FIG. 1, it should be appreciated that multiple model scores 112 may be associated with a particular biological sequence as an output generated by machine learning model 108 as aspects of the techniques described herein may be implemented to various outputs of machine learning models used for biological sequence design. According to some embodiments, model scores 112 may correspond to estimates provided by machine learning model 108 associated with performance of the biological sequences 110. In some embodiments, model scores 112 include estimated values indicative of predicted performance of the biological sequences 110. In some embodiments, model scores 112 include point estimates obtained using a point estimation technique. For example, in embodiments where machine learning model 108 includes a regression model, model scores 112 may include point estimates generated as an output of the regression model.

Biological sequences 110 may include a number of biological sequences, e.g., between approximately 100 to approximately 10,000,000. In some embodiments, biological sequences 110 may include a number of biological sequences in a range of 100 to 100,000, 100 to 200,000, 100 to 500,000, 100 to 1,000,000, 100 to 10,000,000, 100,000 to 10,000,000, or any value or range of values in the range of 100 to 10,000,000. Biological sequences 110 may include biological sequences generated by making one or more mutations (e.g., substitutions, deletions, insertions) from a starting sequence (e.g., wild-type sequence, one of biological sequences 104). In some embodiments, individual biological sequences 110 may have between 1 and 3 mutations, between 1 and 5 mutations, between 1 and 10 mutations, between 1 and 20 mutations, between 1 and 50 mutations. In some embodiments, biological sequences 110 include nucleic acid sequences (e.g., deoxyribonucleic acid (DNA), ribonucleic acid (RNA), and different types of RNA, such as messenger RNA (mRNA)). In some embodiments, biological sequences 110 include amino acid sequences (e.g., a polypeptide sequence, a region of a polypeptide sequence for a protein). Biological sequences 110 may have any suitable length (e.g., number of nucleotides for a nucleic acid sequence, number of amino acids for a polypeptide sequence).

According to some embodiments, biological sequences 104 in training data 102 and biological sequences 110 generated by machine learning model 108 may both be nucleic acid sequences or amino acid sequences. In some embodiments, biological sequences 104 in training data 102 and biological sequences 110 generated by machine learning model 108 may both include biological sequences corresponding to the same region of a protein. In such embodiments, biological sequences 104 in training data 102 and biological sequences 110 generated by machine learning model 108 may both include biological sequences having a similar number of amino acids or a similar number of nucleotides.

Some embodiments involve obtaining measurements 116 for biological sequences 110 generated by machine learning model 108. As shown in FIG. 1, measurement 116a is associated with biological sequence 110a, measurement 116b is associated with biological sequence 110b, measurement 116c is associated with biological sequence 110c, . . . measurement 116n is associated with biological sequence 110n.

According to some embodiments, the techniques described herein for predicting performance of biological sequences designed using a machine learning model are implemented to generate predictions for an attribute of the biological sequences. In such embodiments, the labels for the biological sequences of the training data used in training the machine learning model and the model scores associated with the biological sequences generated by the machine learning model both correspond to the attribute. In FIG. 1, labels 106 for biological sequences 104 may correspond to the same attribute as model scores 112 for biological sequences 110 generated by machine learning model 108. In some embodiments, labels 106 include measurements for the attribute of biological sequences 104. The measurements may be obtained using any suitable technique, including any biological, clinical, toxicology, screening or other assays or animal models (e.g., mouse, non-human primate). These measurement techniques may also be used in obtaining measurements 116 for biological sequences 110.

Examples of attributes include protein binding affinity, protein fluorescence level, protein structure, and production efficiency (e.g., protein folding accuracy). Some embodiments may involve using the techniques described herein in designing viral capsids, e.g., dependoparovirus capsid proteins, adeno-associated dependoparovirus (AAV) capsid proteins, or a region of a viral capsid. In such embodiments, examples of attributes include production efficiency, packaging efficiency, transduction efficiency for a target tissue (e.g., ocular tissue, muscle tissue, brain tissue, liver tissue, kidney tissue), biodistribution efficiency for a target tissue (e.g., ocular tissue, muscle tissue, brain tissue, liver tissue, kidney tissue).

An attribute of interest may depend on the goal in designing biological sequences using a machine learning approach and the type of biological sequences being designed. As an example, a goal in designing protein sequences may involve improving protein-protein binding interactions where the attribute of interest is protein affinity and increasing protein affinity is a desired outcome in design of protein variants. In such an example, training data 102 may include an initial design round of amino acid sequences and associated labels that include measurements for protein affinity for these amino acid sequences, e.g., measurements obtained by performing a binding screening assay using proteins having these amino acid sequences. Model scores 112 may include estimates for protein affinity of amino acid sequences generated by machine learning model 108. As another example, viral capsids (e.g., dependoparoviruses) that produce well (e.g., at a similar level to a wild-type viral capsid) may be an important design goal, and here the attribute of interest is packaging efficiency rate of variant viral capsids (e.g., variants of a wild-type dependoparovirus capsid proteins). In such an example, training data 102 may include amino acid sequences for a region of the viral capsids (e.g., a variable loop region) and associated labels that include measurements for packaging efficiency for viral capsids comprising these amino acid sequences.

According to the techniques described herein, statistical model 118 is configured to allow for at least some predictions for an attribute of biological sequences 110 to occur outside a distribution of labels 106 in training data 102. Using these techniques, statistical model 118 may account for improved performance predictions 120 for biological sequences 110, particularly those biological sequences that are top performing (e.g., biological sequences represented by the right side of distribution 122). In this manner, statistical model 118 may account for “label shift” as biological sequences 104 in training data 102 are often lower performing than biological sequences 110 generated, as represented by shift along the y-axis of between training data label distribution 124 and predicted distribution of labels 122. In some embodiments, statistical model 118 allows for at least some of predictions 120 to occur outside a range of the distribution of labels 124 in the training data. In such embodiments, a range of distribution may correspond to the range of values of the attribute for the distribution (e.g., the range of “y” values for the predicted distribution of labels 122). According to some embodiments, statistical model 118 is configured to allow for at least some of predictions 120 to occur outside a distribution of model scores generated by ensembling the model scores. FIG. 3 is a schematic showing the distribution of model scores 202, which may be obtained by ensembling model scores generated by a machine learning model (e.g., model scores 112 generated by machine learning model 108). FIG. 4 is a schematic showing mode 400b of the predicted distribution of labels occurs outside the distribution of the distribution of model scores 202 shown in FIG. 3. Both FIG. 3 and FIG. 4 include the distribution of labels 200 in the training data as reference.

Some embodiments involve using statistical model 118 and model scores 112 to identify estimates for parameters (e.g., mean, variance) of a probability distribution for biological sequences 110. The probability distribution for biological sequences 110 may represent a distribution of predicted values for an attribute of interest during design of biological sequences 110 (e.g., the attribute associated with labels 106, the attribute associated with model scores 112). In this way, the probability distribution for biological sequences 110 may be considered to represent the “population-level” performance of biological sequences 110. In some embodiments, a probability distribution is determined for individual biological sequences 110 (e.g., a probability distribution for each of biological sequences 110). Here, the probability distribution for a particular biological sequence may be considered to represent the “sequence-level” performance of that particular biological sequence. These “sequence-level” probability distributions may be used to generate a “population-level” probability distribution. In some embodiments, the “sequence-level” probability distributions are used to generate predictions 120, which may include a prediction of a biological sequence's expected value for an attribute. In some embodiments, predictions 120 are generated by sampling values for the attribute using the “sequence-level” probability distributions, and aggregating the sampled values to form a “population-level” probability distribution.

In some embodiments, statistical inference techniques are used to infer a posterior distribution for labels of biological sequences 110, which may be considered as “population-level” posterior distribution. Such embodiments may involve using the statistical inference techniques to identify, using the model scores, estimates for parameter(s) of the posterior distribution for labels of biological sequences 110. In some embodiments, inferring the “population-level” posterior distribution for labels may involve identifying, using model scores 112, estimates for parameters of a posterior distribution for each of biological sequences 110, which may be considered as “sequence-level” posterior distribution. In such embodiments, a “sequence-level” posterior distribution may be generated for a biological sequence by using statistical inference techniques and the model score(s) associated with the biological sequence to identify estimates for parameters of a posterior distribution for the biological sequence. In this way, a set of “sequence-level” posterior distributions may be generated for biological sequences 110. Generating a “population-level” posterior distribution for biological sequences 110 may involve sampling from the “sequence-level” posterior distributions, which may include simulating instances of labels for biological sequences 110.

In some embodiments, statistical model 118 is used to identify estimates for parameters of the probability distribution for each of biological sequences 110 based on model scores 112. Identifying the parameters may involve identifying means and variances for the model scores, where each mean and each variance corresponds to one biological sequences of the biological sequences 110. In some embodiments, statistical model 118 comprises a multimodal model (e.g., a Gaussian mixture model) having a first mode and a second mode, and identifying estimates for the parameters may involve identifying a first set of estimates for parameters associated with the first mode and a second set of estimates for parameters associated with the second mode. One mode may be associated with “non-functional” or “broken” biological sequences and the other mode may be associated with “functional” sequences. The first set of estimates for parameters associated with the first mode may be used to generate a predicted distribution of labels associated with the first mode. The second set of estimates for parameters associated with the second mode may be used to generate a predicted distribution of labels associated with the second mode. The predicted distribution of labels associated with the first and second modes may be used to generate the predicted distribution of labels 122.

According to some embodiments, regression techniques are used to identify estimates for parameters of a probability distribution. In some embodiments, isotonic regression techniques are used to identify estimates for parameters of a probability distribution. Thus, in some embodiments, statistical model 118 may include one or more regression models (e.g., isotonic regression models). In embodiments where statistical model 118 comprises a multimodal model having a first mode associated with “non-functional” biological sequences and a second mode associated with “functional” biological sequences, statistical model 118 may include a first regression model trained on biological sequences and labels associated with the first mode and a second regression model trained on biological sequences and labels associated with the second mode. Identifying the estimates for parameters of the probability distribution may involve using the first regression model to identify a first set of estimates for parameters associated with the first mode and using the second regression model to identify a second set of estimates for parameters associated with the second mode.

According to some embodiments, statistical model 118 may allow for covariate shift by applying a correction to the predictions related to a sequence distance metric (e.g., edit distance). The sequence distance metric may relate to the amount of mutations relative to wildtype one or more biological sequences. Edit distance is one example of sequence distance metric. Another example of a sequence distance metric is BLOSUM, which is described further in S. Henikoff and J. Henikoff, “Performance evaluation of amino acid substitution matrices,” Proteins: structure, Function, and Bioinformatics, 17(1):49-61, 1993, which is incorporated herein by reference in its entirety. In some embodiments, statistical model 118 may include one or more parameters related to a sequence distance metric (e.g., edit distance) for the biological sequences, and using statistical model 118 to generate an output indicating a predicted distribution of labels may comprise identifying an estimate for a parameter by using the sequence distance metric (e.g., edit distance). In some embodiments, using statistical model 118 to generate predictions 120 may involve applying the estimate for the parameter identified using the sequence distance metric as adjust one or more predicted values. In some embodiments, generating the output indicating the predicted distribution of labels for the attribute may involve adjusting the predicted distribution of labels based on the estimate for the parameter identified using the sequence distance metric.

Techniques for predicting performance of biological sequences may be implemented to aid decision-making in the biological sequence design setting. In some embodiments, a performance metric may be generated based on the output indicating the predicted distribution of labels. Some embodiments involve using the output indicating the predicted distribution of labels to determine a likelihood of biological sequences comprising one or more biological sequences having a measurement for an attribute (e.g., the same attribute for labels 106) greater than the labels in the training data. Some embodiments involve using the output indicating the predicted distribution of labels to determine a number of biological sequences from among biological sequence 110 as having a value for an attribute (e.g., the same attribute for labels 106) above a threshold value. According to some embodiments, a performance metric for biological sequences 110 generated using the output indicating the predicted distribution of labels may be used to evaluate whether to manufacture the biological sequences 110 generated by machine learning model 108.

Some embodiments may involve selecting, based on the predicted distribution of labels for an attribute of biological sequences 110, a subset of biological sequences 110. Some embodiments further involve manufacturing the subset of biological sequences. Some embodiments further involve obtaining measurements for the subset of biological sequences.

Some embodiments may involve evaluating, based on the performance metric, whether to obtain measurements for biological sequences 110, including measurements for an attribute for biological sequences 110. Based on evaluating whether to manufacture biological sequences 110 or obtain measurements for biological sequences 110, a decision may be reached as to whether redesign of additional biological sequences is necessary. Accordingly, some embodiments may involve generating a second set of biological sequences at least in part by using machine learning model 108 to obtain the second set of biological sequences and a second set of biological sequences. Statistical model 118 may be used to generate a second output indicating a predicted distribution of labels for an attribute of the second set of biological sequences, and biological sequences 110 or the second set of biological sequences may be selected based on the output indicating the predicted distribution of labels for biological sequences 110 and the second output. Some embodiments involve manufacturing biological sequences 110 or the second set of biological sequences based on the selection. Some embodiments involve obtaining measurements for biological sequences 110 or the second set of biological sequences based on the selection.

Some embodiments may involve training machine learning model 108 on a subset of training data 102 or a different set of training data, and using the trained machine learning model to generate a second set of biological sequences. Statistical model 118 may be used to generate a second output indicating a predicted distribution of labels for an attribute of the second set of biological sequences, and biological sequences 110 or the second set of biological sequences may be selected based on the output indicating the predicted distribution of labels for biological sequences 110 and the second output. Some embodiments involve manufacturing biological sequences 110 or the second set of biological sequences based on the selection. Some embodiments involve obtaining measurements for biological sequences 110 or the second set of biological sequences based on the selection.

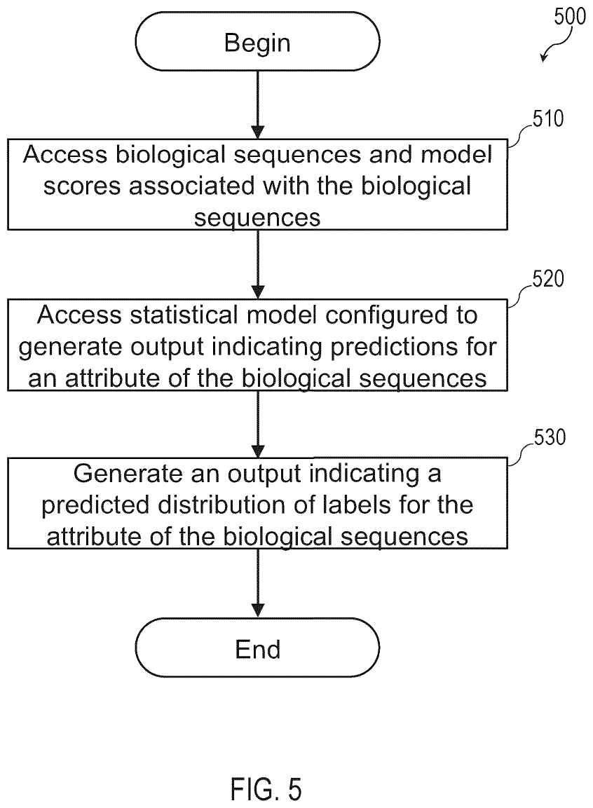

FIG. 5 is a flow chart of an illustrative process 500 for predicting performance of machine-guided designed biological sequences, in accordance with some embodiments of the technology described herein. Process 500 may be performed on any suitable computing device(s) (e.g., a single computing device, multiple computing devices co-located in a single physical location or located in multiple physical locations remote from one another, one or more computing devices part of a cloud computing system, etc.), as aspects of the technology described herein are not limited in this respect.

Process 500 begins at act 510, where biological sequences and model scores associated with the biological sequences are accessed. The biological sequences and model scores are generated using a machine learning model trained on training data comprising biological sequences and labels for an attribute of the biological sequences. In some embodiments, the biological sequences are polypeptide sequences. In some embodiments, the biological sequences are nucleotide sequences.

The machine learning model have any architecture suitable for generating biological sequences. In some embodiments, the machine learning model includes a convolutional neural network. In some embodiments, the machine learning model includes a recurrent neural network. In some embodiments, the machine learning model includes a multi-layer perception. In some embodiments, the machine learning model includes a random forest model. In some embodiments, the machine learning model includes a regression model. Examples of regression models include linear regression models (e.g., simple linear regression, multiple linear regression), polynomial regression models, and Bayesian linear regression models. In some embodiments, the regression model has a convolutional neural network architecture. An example of an algorithm that implements machine learning model(s) suitable for designing biological sequences and may be used to generate biological sequences and model scores 112 is CMA-ES, which is described in N. Hansen and A. Ostermeier, “Completely derandomized self-adaption in evolution strategies,” Evolutionary computation, 9(1):159-195, 2001. Another example of an algorithm that implements machine learning model(s) suitable for designing biological sequences and may be used to generate biological sequences and model scores is CbAS, which is described in D. Brookes, H. Park, and J. Listgarten, “Conditioning by adaptive sampling for robust design,” arXiv preprint arXiv:1901.10060, 2019. Another example of an algorithm that implements machine learning model(s) suitable for designing biological sequences and may be used to generate biological sequences and model scores is Adalead, which is described in S. Sinai, R. Wang, A. Whatley, S. Slocum, E. Locane, and E. Kelsic, “Adalead: A simple and robust adaptive greedy search algorithm for sequence design,” arXiv preprint arXiv:2010.02141, 2020.

In some embodiments, the model scores include at least one model score associated with each of the biological sequences. In embodiments where machine learning model includes a regression model, the model scores include regression estimates associated with the biological sequences.

In some embodiments, the biological sequences comprise protein sequences. In some embodiments, the biological sequences comprise polypeptide sequences. Examples of attributes include protein binding affinity, protein fluorescence level, protein structure, and production efficiency (e.g., protein folding accuracy). In some embodiments, the biological sequences comprise sequences for dependoparvovirus capsid proteins. In some embodiments, the dependoparvovirus is an adeno-associated dependoparvovirus (AAV). In some embodiments, the biological sequences comprise variants of a wild-type dependoparvovirus capsid protein. Examples of attributes of viral capsids (e.g., AAVs, variants of a wild-type dependoparovirus capsid protein) include production efficiency, packaging efficiency, transduction efficiency for a target tissue (e.g., ocular tissue, muscle tissue, brain tissue, liver tissue, kidney tissue), biodistribution efficiency for a target tissue (e.g., ocular tissue, muscle tissue, brain tissue, liver tissue, kidney tissue). In some embodiments, the attribute is transduction efficiency for a target tissue type, and the labels comprise values of transduction efficiency for dependoparvovirus capsid proteins. In some embodiments, the attribute includes packaging efficiency, and the labels comprise values of packaging efficiency for the dependoparvovirus capsid proteins.

Nexi, process 500 proceeds to act 520, where a statistical model configured to generate output indicating predictions for the attribute of the biological sequences is accessed. The statistical model is configured to allow for at least some of the predictions to occur outside a distribution of labels in the training data. In some embodiments, the statistical model allow for at least some of the predictions to occur outside a range of the distribution of labels in the training data. In some embodiments, the statistical model is configured to allow for at least some of the predictions to occur outside a distribution of model scores generated by ensembling the model scores.

In some embodiments, the statistical model comprises a multimodal model having a first mode and a second mode. In some embodiments, the statistical model includes Gaussian mixture model(s) comprising a first mode and a second mode. Further examples of Gaussian mixture models that may be implemented according to the techniques described here are described in Sections A.2 and A.3.

In some embodiments, the statistical model includes a first regression model trained on biological sequences and labels associated with a first mode and a second regression model trained on biological sequences and labels associated with a second mode.

In some embodiments, the statistical model includes a parameter relating to a sequence distance metric (e.g., edit distance).

Next process 500 proceeds to act 530, where an output indicating a predicted distribution of labels for the attribute of the biological sequences is generated using the statistical model, the plurality of biological sequences, and the model scores. In some embodiments, the predicted distribution of labels of the attribute comprises a distribution of values corresponding to predictions of the attribute for the biological sequences.

In some embodiments, generating the output using the statistical model, the biological sequences, and the model scores further comprises: identifying, using the model scores, an estimate for at least one parameter of a probability distribution for the biological sequences.

In some embodiments, generating the output using the statistical model, the biological sequences, and the model scores further comprises: determining, for each of the biological sequences, a probability distribution. In some embodiments, determining the probability distribution for each of the plurality of biological sequences further comprises identifying estimates for parameters of the probability distribution for each of the plurality of biological sequences based on the model scores. In some embodiments, identifying parameters of the probability distribution for each of the biological sequences further comprises identifying means and variances for the model scores, each mean and each variance corresponding to one biological sequence of the biological sequences. In some embodiments, determining the probability for each of the biological sequences further comprises determining a posterior distribution for each of the biological sequences and identifying estimates for parameters of the posterior distribution for each of the biological sequences based on the model scores. In embodiments where the statistical model comprises a multimodal model having a first mode and a second mode, and identifying estimates for parameters of the probability distribution for each of the biological sequences further comprises identifying a first set estimates for parameters associated with the first mode and a second set of estimates for parameters associated with the second mode.

In embodiments where the statistical model includes a first regression model trained on biological sequences and labels associated with a first mode and a second regression model trained on biological sequences and labels associated with a second mode, identifying estimates for parameters of the probability distribution further comprises using the first regression model to identify the first set of estimates for parameters associated with the first mode and using the second regression model to identify the second set of estimates for parameters associated with the second mode. In some embodiments, generating the output indicating the predicted distribution of labels for the attribute of the biological sequences further comprises using the first set of estimates for parameters associated with the first mode to generate a predicted distribution of labels associated with the first mode and using the second set of estimates for parameters associated with the second mode to generate a predicted distribution of labels associated with the second mode.

In embodiments where the statistical model includes a parameter relating to a sequence distance metric, generating the output indicating the predicted distribution of labels further comprises using an estimate for the parameter relating to a sequence distance metric to adjust the predictions generated by the statistical model.

In some embodiments, process 500 further comprises manufacturing at least some of the biological sequences. In some embodiments, process 500 further comprises: selecting, based on the predicted distribution of labels for the attribute, a subset of the biological sequences; and manufacturing the subset of biological sequences.

In some embodiments, process 500 further comprises determining, using the output indicating the predicted distribution of labels, a likelihood of the biological sequences comprising at least one biological sequence having a measurement for the attribute greater than the labels. In some embodiments, process 500 further comprises determining, using the output indicating the predicted distribution of labels, a number of biological sequences from among the biological sequences as having a value for the attribute above a threshold value.

In some embodiments, process 500 further comprises evaluating, based on the output indicating the predicted distribution of labels, whether to manufacture the biological sequences or a subset of the biological sequences. In some embodiments, process 500 further comprises evaluating, based on the output indicating the predicted distribution of labels, whether to obtain measurements for the attribute of the plurality of biological sequences or a subset of the biological sequences. In some embodiments, the biological sequences is a first set of biological sequences, and process 500 further comprises: generating, based on the output indicating the predicted distribution of labels, a second set of biological sequences at least in part by using the machine learning model to obtain as output the second set of biological sequences.

In some embodiments, the biological sequences is a first set of biological sequences, the model scores is a first set of model scores, and the output is a first output, and process 500 further comprises: accessing a second set of biological sequences and a second set of model scores associated with the second set of biological sequences; generating, using the statistical model, the second set of biological sequences, and the second set of model scores, a second output indicating a predicted distribution of labels for the attribute for the second set of biological sequences; and selecting the first set of biological sequences or the second set of biological sequences based on the first output and the second output. In some embodiments, process 500 further comprises manufacturing, based on the selecting, the first set of biological sequences or the second set of biological sequences.

In some embodiments, process 500 further comprises an act of outputting an indication of the first output, such as to a user via a user interface.

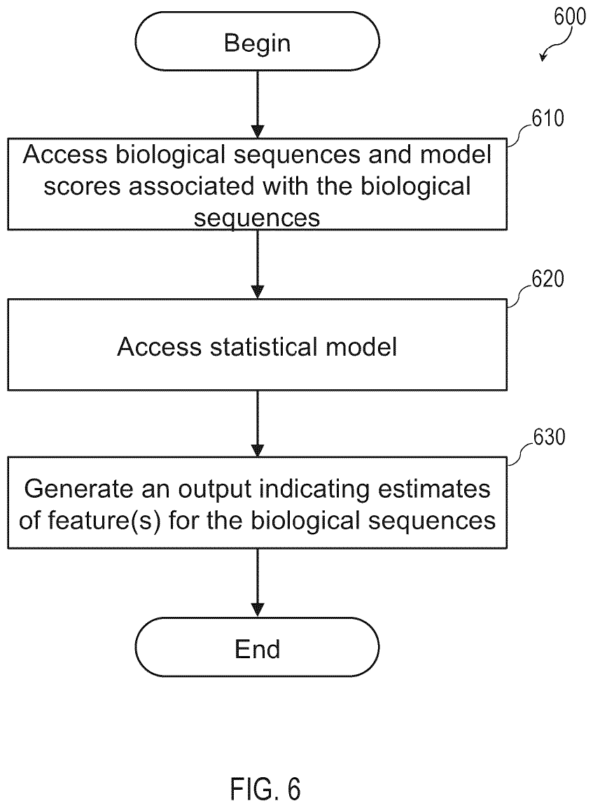

FIG. 6 is a flow chart of an illustrative process 600 for predicting features of machine-guided designed biological sequences, in accordance with some embodiments of the technology described herein. Process 600 may be performed on any suitable computing device(s) (e.g., a single computing device, multiple computing devices co-located in a single physical location or located in multiple physical locations remote from one another, one or more computing devices part of a cloud computing system, etc.), as aspects of the technology described herein are not limited in this respect.

Process 600 begins at act 610, where first biological sequences and a first set of model scores associated with the first biological sequences are accessed. In some embodiments, the first set of model scores include regression estimates associated with the first biological sequences. In some embodiments, the first set of model scores include model scores associated with each of the first biological sequences.

In some embodiments, the first biological sequences comprise protein sequences. In some embodiments, the first biological sequences comprise sequences for dependoparvovirus capsid proteins. In some embodiments, the dependoparvovirus is an adeno-associated dependoparvovirus (AAV). In some embodiments, the first biological sequences comprise variants of a wild-type dependoparvovirus capsid protein. In some embodiments, the feature(s) include transduction efficiency for a target tissue type. In some embodiments, the feature(s) include packaging efficiency.

Nexi, process 600 proceeds to act 620, where a statistical model configured to generate output indicating estimates for feature(s) of a biological sequence is accessed. In some embodiments, the statistical model includes Gaussian mixture model(s). Further examples of Gaussian mixture models that may be implemented according to the techniques described here are described in Section A.2 and A.3. In some embodiments, one or more parameters of the statistical relates a calibration value for estimates of the feature(s) based on edit distance of a biological sequence to a wildtype biological sequence.

In some embodiments, the statistical model was trained using training data that includes a second set of model scores associated with second biological sequences and measurement data for the second biological sequences. In some embodiments, at least some of the estimates have values greater than values of the measurement data. In some embodiments, at least some of the estimates have values greater than a highest value of the measurement data. In some embodiments, some of the first biological sequences having a model score greater than a threshold value are estimated to have a value for the feature(s) greater than for the second biological sequences.

According to some embodiments, process 600 may comprise training the statistical model using training data that includes a second set of model scores and measurement data for second biological sequences. In some embodiments, training the statistical model further comprises identifying parameter(s) of the statistical model using the second set of model scores and the measurement data for the second biological sequences. Identifying the parameter(s) may comprise the acts of: identifying means and variances for the second set of model scores, identifying means and variances for the measurement data for the second biological sequences, and identifying the parameter(s) based on the means and variances for the second set of model scores and the means and variances for the measurement data. In some embodiments, identifying the parameter(s) further comprises using at least one isotonic regression model to identify the parameter(s).

Next process 600 proceeds to act 630, where a first output indicating estimates of the feature(s) for the first biological sequences is generate using the statistical model, the first biological sequences, and the first set of model scores. In some embodiments, the first output includes a distribution of values corresponding to the estimates of the feature(s) for the first plurality of biological sequences. In embodiments where the first biological sequences comprise sequences for dependoparvovirus capsid proteins and the feature(s) include transduction efficiency, the estimates include values of transduction efficiency for dependoparvovirus capsid proteins. In embodiments where the first biological sequences comprise sequences for dependoparvovirus capsid proteins and the feature(s) include packaging efficiency, the estimates include values of packaging efficiency for the dependoparvovirus capsid proteins.

In embodiments where the first set of model scores include model scores associated with each of the first biological sequences, generating the first output further comprises identifying means and variances for the first set of model scores. Each mean and each variance corresponding the model scores associated with one of the first biological sequences.

In embodiments where the statistical model includes Gaussian mixture model(s), generating the first output further comprises sampling distributions of a first feature for the first biological sequences using the Gaussian mixture model(s) and identifying estimates of the first feature based on the distributions. In some embodiments, the sampling further comprises sampling, using the Gaussian mixture model(s), a distribution of the first feature for each of the first biological sequences and identifying estimates of the first feature based on the distributions. In some embodiments, the sampling further comprises sampling a distribution of the first feature for each of the first biological sequences using the Gaussian mixture model(s).

Some embodiments involve using the estimates of the feature(s) for selecting biological sequences to subsequently manufacture and, in some embodiments, experimentally validate. In some embodiments, process 600 further comprises selecting, based on the estimates of the feature(s), a subset of the first biological sequences, and manufacturing the subset of the first biological sequences.

In some embodiments, process 600 further comprises accessing second biological sequences and a second set of model scores associated with the second biological sequences, and generating a second output indicating estimates of the feature(s) for the second biological sequences. Generating the second output involves using the statistical model, the second biological sequences, and the second set of model scores. Process 600 may further comprise the act of selecting the first biological sequences or the second biological sequences based on the first output or the second output. In some embodiments, the method further comprises manufacturing, based on the selecting, the first biological sequences or the second biological sequences.

In some embodiments, process 600 further comprises an act of outputting an indication of the first output, such as to a user via a user interface.

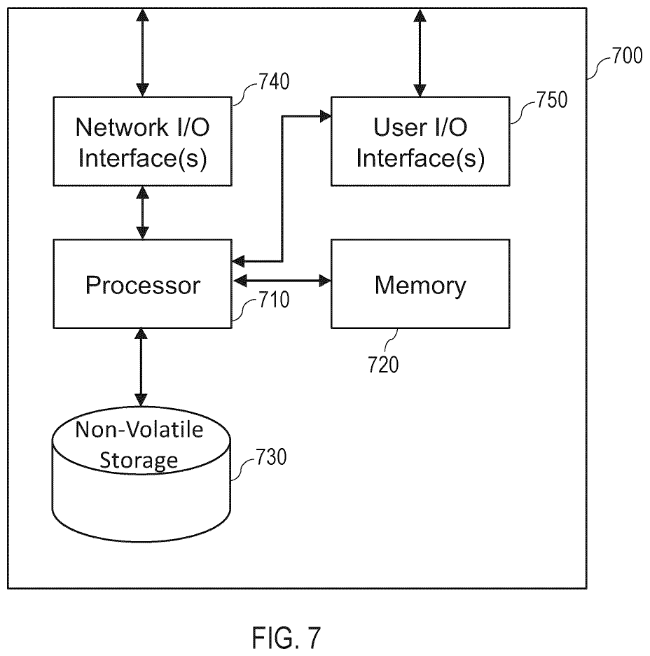

An illustrative implementation of a computer system 700 that may be used in connection with any of the embodiments of the technology described herein is shown in FIG. 7. The computer system 700 includes one or more processors 710 and one or more articles of manufacture that comprise non-transitory computer-readable storage media (e.g., memory 720 and one or more non-volatile storage media 730). The processor 710 may control writing data to and reading data from the memory 720 and the non-volatile storage device 730 in any suitable manner, as the aspects of the technology described herein are not limited in this respect. To perform any of the functionality described herein, the processor 710 may execute one or more processor-executable instructions stored in one or more non-transitory computer-readable storage media (e.g., the memory 720), which may serve as non-transitory computer-readable storage media storing processor-executable instructions for execution by the processor 710.

Computing device 700 may also include a network input/output (I/O) interface 740 via which the computing device may communicate with other computing devices (e.g., over a network), and may also include one or more user I/O interfaces 750, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

Some aspects of the technology described herein may be understood further based on the non-limiting illustrative embodiments described below in Section A. Any limitations of the embodiments described below in Section A are limitations only of the embodiments described in Section A, and are not limitations of any other embodiments described herein.

### Section A

The ability to design and optimize biological sequences with specific functionalities would unlock enormous value in technology and healthcare. In recent years, machine learning-guided sequence design has progressed this goal significantly, though validating designed sequences in the lab or clinic takes many months and substantial labor. It is therefore valuable to assess the likelihood that a designed set contains sequences of the desired quality (which often lies outside the label distribution in our training data) before committing resources to an experiment. Forecasting may be a useful concept in situations where feedback can be delayed, such as in the context of sequence design and evaluation. Here we propose a method to guide decision-making that forecasts the performance of high-throughput libraries (e.g., containing 10⁵ unique variants) based on estimates provided by models, providing a posterior for the distribution of labels in the library. We show that our method outperforms baselines that naively use model scores to estimate library performance, which are the only tool available today for this purpose.

### A.1 Introduction

Biological sequence design has long been of interest to practitioners in many domains, from agriculture to therapeutics. For decades, sequences were designed through two means: (i) labor-intensive rational design where expert human knowledge would generate a handful of candidate sequences; (ii) high-throughput directed evolution approaches that utilize biological evolution to optimize sequences towards a desired property. Recently, the ability to synthesize DNA in high-throughput, together with the wide adoption of high-capacity of machine learning models, has opened a new path that can combine the benefits of rational design (high quality), and directed evolution (high-throughput). In this setting, libraries containing up to 10⁵ sequences are designed using machine learning algorithms. Machine learning methods are used to score, optimize, and filter sequences before committing to experiments. One increasingly nuanced issue is how to improve our trust in the output of machine learning models and paired optimization procedures. Using these methods, sequences targeting different objectives can be synthesized (e.g. transcription factor binding or other regulatory sequences) in a library that can be measured in the desired context. However, especially with products or traits of high complexity (e.g., in-vivo studies of proteins), the overall cost required to validate designs can be prohibitive.

Therefore, even with model evaluations and calibration of uncertainty around samples, there remains a gap in our ability to forecast the probability of success: be it reaching a certain maximum performance, or finding a certain number of variants above a minimum desired performance. This is distinct from attempting to predict the performance of single sequences, in that it focuses on predicting the right-tail distribution of the performance our entire library. In many settings, we would want to know whether the experiment has a high chance of finding a (generally rare) high-performing sequence overall. Forecasts can help us decide whether to commit to a certain design and can save large costs. Forecasts can also inform other decisions such as deciding whether to repeat the design procedure for a library, deciding among libraries designed for different targets, or estimating the final price of developing a drug.

Forecasting may be used in domains with delayed feedback such as elections. The related topic of label shift classically relies on the “anticausal” assumption that the distribution of inputs given labels is constant across training and test sets—an assumption that is invalid in the case of design. More generally, domain adaptation has been studied in biological sequence design but does not directly address forecasting and calibrating distributions under covariate and label shift. To our knowledge, there are currently no methods that are suitable for forecasting library performance in the sequence design setting. This setting presents an interesting and somewhat unique challenge. For every designed sequence we can obtain scores for the expected performance, possibly from multiple models. However we are often aiming to make sequences that have a significantly higher score than anything observed in our training data, i.e. distribution shift is by design. Our challenge is to find the right balance between trusting our models' predictions out-of-distribution and betting that our new designs would provide us with better-than-observed sequences.

### A.2 Forecasting Method Overview

We start with labeled training data (S⁰, Y⁰), where S⁰={sᵢ⁰} is a set of biological sequences and Y⁰={yᵢ⁰} is a set of continuous-valued labels, generally a fitness measurement in the sequence design setting such as packaging or transduction efficiency rates. Our goal is to forecast a distribution of labels Y¹ for an unlabeled set S¹. That is, we are not concerned with the accuracy of each pair (sᵢ¹, yᵢ¹), but only the overall distribution of Y¹, and in particular, in the right tail of Y¹, which indicates the maximum quality achieved by the set of sequences. To create our forecast, we have at our disposal a set of J regression models trained on (S⁰, Y⁰), which produce test set predictions mᵢⱼ for sequence i by model j.

Naively, we could form ensembled point estimates ŷᵢ=Σⱼ mᵢⱼ/J for each sequence and predict the distribution of Y¹ to be the distribution of fj. There are three main disadvantages to this approach, which inspire different aspects of our forecasting method. We address them briefly below, and give a more thorough treatment with a complete algorithm in Section A.3.

1) An implicit unimodal Gaussian assumption Empirically, model ensembles tend towards unimodal Gaussian score distributions which do not empirically fit experimental data from designed sequences well. At several points in the experimental pipeline variants may “drop out,” failing to produce enough signal to reliably approximate a label (for example, due to failure of a protein to fold). This results in a multimodal distribution at both the population level, and implicitly, at the level of each sequence's posterior. Thus, we seek to model each sequence as a bimodal Gaussian Mixture Model (GMM), and learn the parameters for each sequence's posterior from its model scores. Explicitly, given an independently and identically distributed (i.i.d.) set of model predictions mᵢⱼ for sequence i, we seek to infer a probability pᵢ that distinguishes between the two distributions in the Gaussian mixture, as well as a mean and variance parameter for each Gaussian mode. Moreover, while we use a GMM, our method could in theory be applied using a range of more complex distributions, with the only constraint being our ability to sample from them.

2) Distribution (covariate and label) shift Typically, the sequence set S¹ is designed with model-guided exploration strategies informed by (S⁰, Y⁰), with the objective of producing sequences that outperform the best sequences in S⁰. This results in both significant distribution (covariate) shift, because the sequences S¹ are reaching into untested areas of sequence space, and label shift, since we anticipate that Y¹ will dominate Y⁰, both on average and among each sets top-performers. To address distribution and label shift, we start by applying non-parametric non-linear calibration techniques to produce a “conservative” forecast that still allows for some label shift due to model uncertainty. We then consider scenarios with some trust placed in raw model scores to allow for some amount of extrapolation to regions further from our training set.

3) Point estimates to posteriors The point estimates that arise from model ensembles do not provide a posterior for Y¹ (nor do the model score variances, directly), and consequently these tend to underestimate the frequency of events that are rare at the sequence level, but common at the population level, such as the occurrence of high-valued sequences in the library. In our method, we simulate draws of the entire library from the sequence-level posteriors to produce both expected distributions as well as the frequency of rare events, which we interpret as posterior probabilities.

### A.3 Forecasting Method Description

To generate forecasts, we first transform model predictions for each sequence into parameters for their posterior distributions. We then draw from those sequence-level posterior distributions to form simulations of the library, generating a library-level posterior. This library-level posterior reflects our epistemic uncertainty about the ground-truth performance of each sequence given our model predictions and the aleatoric uncertainty of our measurements given this ground-truth. That is, our predictions are more like prediction intervals than confidence intervals, and we do not generate a posterior for ground-truth values. Our objective is to infer sequence-level posterior parameters from model predictions in a way that is both well-calibrated to the training data and allows for test set performance of the sequences to differ from or exceed training set performance due to distribution shift.

### A.3.1 Fitting Sequence Label Posteriors

While this forecasting framework can be applied to learn posteriors for many distributions families, informed by empirical library label distributions from historical experiments, we believe the natural distribution for sequence labels in our data is a Gaussian mixture model (GMM) with two modes: one for “functional” sequences and one for “non-functional” or “broken” sequences. Other distribution families, including varying the skew and kurtosis of each mode of the GMM, may be applied to tune the model.)

To model a sequence sᵢ using a GMM, we assume there is a probability of functionality pᵢ, a mean and variance in the functional mode (μᵢ⁺, (σᵢ⁺)²), and a mean and variance in the non-functional mode (μᵢ⁻, (σᵢ⁻)²), that parameterize normal distributions N so that follows Equation (1), below.

$${Y}_{i} ∼ {p}_{i} N ( {μ}_{i}^{+} , {σ}_{i}^{+} ) + ( 1 - {p}_{i} ) N ( {μ}_{i}^{-} , {σ}_{i}^{-} ) \qquad Equation ( 1 )$$

In contrast, our predictive models only provide point estimates mᵢⱼ for each sequence We assume that the true, multimodal distribution of each sequence can be summarized with two degrees of freedom (a mean μᵢ and standard deviation σ₁) and that these two parameters independently generate model scores, mixture model parameters, and measurement values. Explicitly, we assume the model predictions mᵢⱼ˜N (μᵢ, σᵢ), so that, given a set of model predictions, we infer μᵢ and σᵢ to be the models' sample mean and variance (μᵢ=1/JΣⱼmᵢⱼ and σᵢ²=1/Σⱼ(mᵢⱼ−μᵢ)²). We further assume there are independent relationships between μᵢ and the set (μᵢ, μᵢ⁺, μᵢ⁻), and between σᵢ and the pair Σᵢ⁺ and Σᵢ⁻.

Since the GMM parameters (pᵢ, μᵢ⁺, μᵢ⁻, σᵢ⁺, σᵢ⁻) are unique to each sequence, we cannot infer them in the usual manner using S⁰ as a training set. Instead, we need to further model and learn the relationship between the pair μᵢ, σᵢ and the GMM parameters. Specifically, we start by identifying the value y_{mid} that we use to separate the two modes of Y. This value can either be set manually, using expert knowledge, or automatically by analyzing the distribution Y⁰. Here, we use a method that finds the separating point that minimizes intra-class variance, provided robust values of y_{mid} on our data. An example of such a method is further described in Nobuyuki Otsu, “A threshold selection method from gray-level histograms”, IEEE transactions on systems, man, and cybernetics, 9(1): 62-66, 1979, which is incorporated herein by reference in its entirety. We can then divide our set S⁰ into two halves across the boundary: S⁰⁺={sᵢ|yᵢ⁰≥y_{mid}} and S⁰⁻={sᵢ|yᵢ⁰<y_{mid}}. This provides us with separate training sets for the functional parameters μᵢ⁺, σᵢ⁺ and the non-functional parameters μᵢ⁻, σᵢ⁻

A.3.2 about Isotonic Regression

We will run isotonic regression to find the best monotonic piece-wise linear fit to this data. Explicitly, isotonic regression operates on a dataset of pairs of scalars {xᵢ, yᵢ} and produces a non-parametric model represented by data-prediction pairs (xᵢ, ŷᵢ) that seek to minimize the least squares error Σᵢ(ŷᵢ−yᵢ)² subject to a monotonicity constraint yᵢ≤yⱼ ∀ᵢ, j s.t. xᵢ<xⱼ. This results in a quadratic program, solvable by sorting yᵢ and iteratively averaging pairs of “violators” of monotonicity, making training efficient and deterministic. An example for such a method is further described in Jerome Friedman and Robert Tibshirani, “The monotone smoothing of scatterplots”, Technometrics, 26(3):243-250, 1984, which is incorporated herein by reference in its entirety.

As a point of notation, we will use the abbreviation IR_{y} to refer to an isotonic regression model trained to predict y defined by the pairs (xᵢ, ŷᵢ), and IR_{y} (xⱼ′) to be the prediction of this model given input xⱼ′. To compute IR_{y} (xⱼ′) on a new data point, first we check to see if xⱼ′=xᵢ for some i and if so we predict the corresponding IR_{y} (xⱼ′)=ŷₜ. Otherwise we sort xᵢ and find a consecutive pair such that xᵢ<xⱼ′<xᵢ₊₁ and predict IR_{y} (xⱼ′) by linearly interpolating between ŷᵢ and ŷᵢ₊₁. If xⱼ′ is less than all xᵢ, or more than all xᵢ, IR_{y}(xⱼ′) is set to the min and max values of ŷᵢ respectively.

A.3.3 Inferring Parameters with Isotonic Regression

We assume a non-linear monotonic relationship between the model ensemble mean for a sequence μᵢ=1/JΣⱼμᵢⱼ and the probability that the sequence i will be functional (pᵢ). To infer pᵢ, we train an isotonic regression model IRₚ that, given μᵢ, aims to predict the indicator Iᵢ⁺ which is 1 if sᵢ>y_{mid} and 0 otherwise. Effectively, given a new input μᵢ, this model returns the fraction of sequences that are functional out of the training samples with similar mean values, and interprets this rate as the probability that the sequence i will be functional.

Inferring the mean parameters μ⁺ and μ⁻ is more straight-forward: we build isotonic regression models IR_{μ}+ and IR_{μ}− to predict yᵢ from μᵢ, but restrict the training set to S⁰⁺ and S⁰⁻ respectively. This gives us calibration to the conditional distributions for being functional and non-functional respectively. To infer the variances σᵢ²⁺ and σᵢ²⁻, we first form squared residuals of labels given model ensemble means resᵢ=(yᵢ−μᵢ)² and build isotonic models IR_{σ}+ and IR_{σ}-relating the model variance to these residuals resᵢ. As with μ⁺ and μ⁻, we compute σᵢ²⁺ and σᵢ²⁻ by training models on the disjoint training sets S⁰⁺ and S⁰⁻ respectively. The complete algorithm for inferring the GMM parameters is described in Algorithm 1.

Applying the forecast to non-ensembles While the presentation of our method assumes access to an ensemble of models, we note that thus far the only information we have used from the ensembles is the ensemble mean and variance (μᵢ, σᵢ²) for each feature. Therefore, as an alternative, any single model that itself outputs an expected value and uncertainty (which includes many neural networks) can stand alone in providing the input (μᵢ, σᵢ²) to forecasting calibration. The only technique that does not generalize from ensembles to models-with-uncertainty is the “optimistic model de-ensembling” technique discussed in the Section A.3.5.

### A.3.4 Simulating the Posterior Distribution

Given the parameters generated by Algorithm 1, we can draw samples ŷᵢ¹ for each sequence, and aggregate them into draws for the entire distribution Ŷ¹. We can then treat the set of simulated values of Ŷ¹ as a posterior distribution and query this distribution to determine the frequency of distribution-level events. By computing metrics on Ŷ¹ and considering their distributions across simulations, we can arrive at empirical confidence intervals for metrics such as the count of sequences that perform above some threshold value, as we see in FIGS. 8B, 8D, 8F, and 8H.

A.3.5 Tuning the Forecast from Conservative to Optimistic

We can further refine this algorithm using additional techniques that allow us to diversify our approach over degrees of trust in our training set.

Semi-calibrated regression Our main calibration tool, isotonic regression, aggressively limits predicted labels to be within the range of training values. To allow for some distribution shift, we can gradually transition from calibrated predictions towards the center of the distribution of S¹ towards uncalibrated, out-of-distribution values towards the limits of the distribution, in a technique we call “semi-calibration.”

Let P_{Y}(y) be the percentile of the value y from among the empirical distribution Y. That is, P(y) is the fraction of y∈Y with yᵢ<y. In our case, we consider the distribution of model ensemble means on our training set S⁰, that is, the set M={1/JΣⱼmᵢⱼ|sᵢ∈S⁰}. Then given a new sequence sᵢ we can compute its model ensemble prediction μᵢ as well as its functional isotonically calibrated mean μᵢ⁺, and evaluate where its model ensemble falls relative to the training distribution by computing the percentile P_{M} (μᵢ). Finally, for any temperature-like coefficient 0<q≤1, we define our semi-calibrated mean \tilde{μ}ᵢ(q) to be Equation (2), below.

$${\tilde{μ}}_{i} ( q ) = ( q {P}_{M} ( {μ}_{i} ) ) ( {μ}_{i} ) + ( 1 - q {P}_{M} ( {μ}_{i} ) ) ( {IR}_{{μ}_{i}} + ( {μ}_{i} ) ) . \qquad Equation ( 2 )$$

Thus, lower values will be completely calibrated to the training set, while higher values will be a mix of calibrated and uncalibrated values. Note that we only produce this correction for functional mean values μ⁺, as we expect non-functional values to be fully in the training set distribution. This leads to an update to the model from Equation 1, as shown in Equation 3, below.

$${\hat{y}}_{i} ∼ {p}_{i} 𝒩 ( {\tilde{μ}}_{i} ( q ) , {σ}_{i}^{+} ) + ( 1 - {p}_{i} ) 𝒩 ( {μ}_{i}^{-} , {σ}_{i}^{-} ) \qquad Equation ( 3 )$$

Correcting for covariate shift In addition to model score distribution shift, we also see covariate shift that creates model score bias. In our contexi, we consider edit distance to wild type the primary such covariate, though the method applied to other distance metrics (such as BLOSUM, which is described further in S. Henikoff and J. Henikoff, “Performance evaluation of amino acid substitution matrices,” Proteins: structure, Function, and Bioinformatics, 17(1):49-61, 1993), as well as other quantitative side-information. To correct for this shift, we form signed residuals from the training set between the calibrated μᵢ⁺ values and the true values yᵢ(i.e. (μᵢ⁺−yᵢ)). (If we are also applying the semi-calibration technique from the last paragraph, we use (\tilde{μ}ᵢ−yᵢ) instead.) We can regress those residuals on edit distance (EDᵢ) using either isotonic or linear regression, and apply this correction back to the mean prediction μᵢ⁺. We can also apply this approach to adjust the probability parameter pᵢ, encoding the understanding that sequences are less likely to be functional at higher distances from the wild-type. That is, we compute Equations (4), (5), (6), and (7).

$$r e {s}_{i} = {μ}_{i}^{+} - {y}_{i} \qquad Equation ( 4 )$$

$${IR}^{E D} = IR model trained on \{ ( E {D}_{i} , re {s}_{i} ) \} \qquad Equation ( 5 )$$

$${μ}_{i}^{E D} = {μ}_{i}^{+} - {IR}^{E D} ( E {D}_{i} ) \qquad Equation ( 6 )$$

$${\hat{y}}_{i} ∼ {p}_{i} 𝒩 ( {μ}_{i}^{E D} , {σ}_{i}^{+} ) + ( 1 - {p}_{i} ) 𝒩 ( {μ}_{i}^{-} , {σ}_{i}^{-} ) \qquad Equation ( 7 )$$

Optimistic model de-ensembling So far, we have assumed model scores mᵢⱼ are drawn from a Gaussian distributions parameterized by μᵢ, σᵢ. As an alternative, we could assume that each model j represents a distinct distribution, and that μᵢ are drawn from these distributions with equal probability. Using this approach, in each simulation we first randomly select one model independently for each sequence and use that model's prediction as the sequence's expected value: μᵢ mᵢⱼ. This can result in more optimistic forecasts when scores have high inter-model variance.

Hedging against calibration assumptions Together, these calibration techniques create a menu of options that allow us to build forecasts that range from conservative to optimistic given input data. Given a set of calibration strategies, we can simulate instances of Y¹, and by aggregating simulations across frameworks, we can form a posterior for the distribution of Y¹ that captures our uncertainties at the sequence level, the model level, and the overall forecasting approach level.

### A.4 Experimental Results

We validate our method by conducting experiments on four datasets—a set of simulated RNA binding landscapes that allow us to access ground truth values for every sequence (and repeat multiple experiments) as well as three experimentally-measured assays of protein fitness landscapes. These are a viral protein packaging landscape, an experimentally measured IgG-Fc binding dataset for Protein G's GB1 domain, and an experimentally measured GFP fluorescence dataset.

### A.4.1 Descriptions of Experimental Data

### A.4.1.1 Simulated RNA Landscapes

Our first set of experiments investigates the performance of forecasting approach using FLEXS, a simulation environment for sequence design which gives access to ground-truth and model-approximated fitness landscapes. FLEXS is described further in S. Sinai, R. Wang, A. Whatley, S. Slocum, E. Locane, and E. Kelsic “Adalead: A simple and robust adaptive greedy search algorithm for sequence design”, arXiv preprint arXiv: 2010.02141, 2020, which is incorporated herein by reference in its entirety. We study design problems on two RNA landscapes with a hidden binding target of size 14 and 50 nucleotides. Each training set S⁰ was constructed by mutating a sequence from a starting seed (5 seeds for each landscape) with between 1 and 3 mutations per sequence on average. We trained four kinds of predictive models on one-hot encoded sequences: linear regressions, convolutional neural networks, multi-layer perceptions, and random forest models. We used four exploration algorithms to design sequences using these models S¹: CMA-ES, CbAS, Adalead, and random sampling. CMA-ES is further described in N. Hansen and A. Ostermeier, “Completely derandomized self-adaption in evolution strategies,” Evolutionary computation, 9(1):159-195, 2001. CbAS is further described in D. Brookes, H. Park, and J. Listgarten, “Conditioning by adaptive sampling for robust design,” arXiv preprint arXiv:1901.10060, 2019. Adalead is further described in S. Sinai, R. Wang, A. Whatley, S. Slocum, E. Locane, and E. Kelsic, “Adalead: A simple and robust adaptive greedy search algorithm for sequence design,” arXiv preprint arXiv:2010.02141, 2020.

### A.1.2 In-Vitro AAV Packaging Assay

A data set obtained by quantitatively assaying the packaging efficiency of 200K viral capsid variants, modified in a 28 amino-acid region of the protein was used. This data set is further described in D. Bryant, A. Bashir, S. Sinai, N. Jain, P. Ogden, P. Riley, G. Church, L. Colwell, and E. Kelsic, “Deep diversification of an AAV capsid protein by machine learning”, Nature Biotechnology, 39(6):691-696, 2021, which is incorporated herein by reference in its entirety. The experiment was designed in two steps, where first a smaller set of training examples were assayed and used to train classification models. Then these models were used to design a set of variants that were optimized for the probability of packaging. Classification models and greedy optimization were used to generate the second batch. The existence of this first training set, and the distribution-shifted designed set is exactly the setting we have devised the forecasting method for, and where its performance can be most meaningfully evaluated.

For this data set we retrain regression models on the first set of sequences (S⁰) using five independently seeded convolutional neural networks, and five recurrent neural networks. We used these models to generate ensembled point estimates as well as forecast the distribution of packaging efficiencies on the model-designed set of sequences (S¹).

### A.4.1.3 Protein G GB1 IgG-Fc Binding Domain

A region of four amino acids in Protein G (GB1) is known to be critical for IgG-Fc binding and has been used extensively as a tool for evaluating sequence landscape prediction and design tasks described in C. Smith and T. Kortemme, “Predicting the tolerated sequences for proteins and protein interfaces using rosettabackrub flexible backbone design”, PloS one, 6(7):e20451, 2011, and S. Kmiecik and A. Kolinski, “Folding pathway of the b1 domain of protein g explored by multiscale modeling”, Biophysical journal, 94(3):726-736, 2008, each of which is incorporated herein by reference in its entirety. Data is sourced from experiments described in N. Wu, L. Dai, C. Olson, J. Lloyd-Smith, and R. Sun, “Adaption in protein fitness landscapes is facilitated by indirect paths”, Elife, 5:e16965, 2016, which is incorporated herein by reference in its entirety. We created S⁰ by selecting sequences with performance below that of the wild-type, combined with a small fraction of sequences between wild-type and the median performance in the set, leaving all other sequences for S¹. We trained five independently seeded random forest models and five multi-layer perceptrons on S⁰ (forgoing more sophisticated models due to the short length of the variable sequence), and used these to generate point estimates and forecast predictions for S¹.

### A.4.1.4 Green Fluorescent Protein

The green fluorescent protein of Aequorea victoria (avGFP) provides an additional fitness landscape for studying sequence prediction and design. We used a dataset of 540,250 protein variants and their associated fluorescence level, which is described in K. Sarkisyan, D. Bolotin, M. Meer, D. Usmanova, A. Mishin, G. Sharonov, D. Ivankov, N. Bozhanova, M. Baranov, O. Soylemex et al, “Local fitness landscape of the green fluorescent protein”, Nature, 533(7603):397-401, 2016, which is incorporated herein by reference in its entirety. We followed the same procedure as GB1 for splitting S⁰ and S¹, putting sequences with fluorescence below 3× log-fluorescence of the wild-type and a small portion of sequences up to the median fluorescence above this threshold into S⁰, and the rest into S¹. We then trained the same models as in our AAV experiments—five independently seeded convolutional neural networks, and five recurrent neural networks, and generated point estimates and forecasts for S¹ using these models.

### A.4.3 Experimental Design

For each of these experiments, we generated training and test sets S⁰, Y⁰ and S¹, Y¹, and models M. We used the training set, models, and unlabeled test data to generate forecasts, and evaluated them against realized distributions of Y¹ using two key tools: a 2-sample Kolmogorov-Smirnov statistic measuring distribution fit of top percentiles, and confidence interval coverage for counts of points measured above a fixed threshold value. Both evaluate fit in the right tail of the distribution, which is both the most challenging region to predict, and the one that is most critical to sequence design applications. We present results of forecasts on all four datasets in FIG. 8, with one dataset per row of figures (RNA in FIGS. 8A and 8B, AAV in FIGS. 8C and 8D, GB1 in FIGS. 8E and 8F, and GFP in FIGS. 8G and 8H). These experiments demonstrate the efficacy of the forecasting procedure in a variety of experimental settings.

In the left column of plots in FIG. 8 (8A, 8C, 8E, 8G) we report on the Kolmogorov-Smirnov fit between the true distribution of scores Y¹ and either the forecast or the set of point estimates μᵢ from model ensemble scores. In FIG. 8A we plot the mean and 95% confidence interval across landscapes and starts (see Sec A.4.1.1 for details), while the other experiments have a single landscape each. Since we are primarily concerned with fit in the right tail, we limited each distribution to values above a percentile threshold, and varied that threshold between 50% and 95%. Across this range, the forecasting method improved distributional fit compared to ensemble point estimates, and while point estimates typically decayed towards 1.0 (the theoretical worst-case upper bound of the statistic), the forecast consistently maintained some predictive power even in the top 5th percentile. A sharp covariate shift in the AAV capsid design sets S⁰ and S¹ account for the problem difficulty in FIGS. 8E and 8F, though even in this problem our method directionally improves upon model estimates.

In the right column of FIG. 8 (FIGS. 8B, 8D, 8F, and 8H) we focus more closely on the right tail of the label distribution of Y¹, reporting the forecast's confidence interval for the number of sequence we can expect to find above a threshold value compared to the estimate from model ensembles and the true counts. Since FIG. 8B encompasses several landscapes and seeds, we set one threshold per landscape/seed at the 99th percentile, while in the remaining experiments with a single landscape we evaluate accuracy over a range of thresholds. Here we see that the forecast gives confidence intervals that include the true count of sequences above a high threshold some of the time, and always improves upon the ensemble estimates, which for most datasets severely underpredicts the prevalence of top-performers.

A key item of interest for sequence design is the performance of the top variants. We report ensemble, forecast, and true values for the 99th percentile, mean of the top percentile, and maximum value for the AAV, GB1, and GFP experiments in FIG. 9. Results from each individual RNA experiment can be found in FIG. 10. These results echo the conclusions from FIG. 8, showing highly accurate predictions on the AAV and GFP landscapes, and directionally correct adjustments on GB1 and RNA landscapes.

### A.5 Conclusion

We've demonstrated the relevance and impact of forecasting in the sequence design setting. We developed a novel approach for forecasting label distributions under covariate and label shift that occurs during model-guided design. Our approach can be used on any machine-guided library design for which we have regression models. We applied these methods in simulated and real-world sequence design settings and showed near-universal improvement (and never worse than the naive approach) in our ability to predict the shape of the right tail and counts of top performers. This work enables valuable estimation of the quality of designed libraries of biological sequences long before experimental results can be produced, which provides essential feedback to the designer.

### A.6 Computational Details

### A.6.1 Compute

All of our experiments were run using a single server with a single GPU running in GCP (Google Cloud Platform). We used an Nvidia V100 for training models on the GFP landscape and an Nvidia K80 for the other three experiments' model training.

### A.6.2 Hyperparameters

Across all of our experiments, we used five model architectures: convolutional neural networks (CNNs), recurrent neural networks (RNNs), multi-layer perceptrons (MLPs), linear models, and random forests. Linear models and random forests were initialized with default parameters using the sklearn library. CNNs used 32 filters, 64 filters, and 256 filters with 1, 2, and 2 convolutional layers followed by 1, 2, and 2 hidden layers of width 32, 64, and 64 for the AAV, RNA, and GFP experiments respectively. RNNs used embeddings of size 32 combined with 1 and 2 recurrent layers, then followed by 1 hidden layer of size 56 and 128 for AAV and GFP respectively. MLPs used 1 and 3 hidden layers of width 50 and 32 for GB1 and RNA experiments respectively. All three model architectures were trained used Adam with a learning rate of 1e-3 across experiments.

### A.6.3 Licenses

FLEXS is open source and Apache licensed. All other code was written for this project in python using common packages that use BSD, PSFL, Apache, and MIT licenses.

### A.7 Additional RNA Results

FIG. 10 shows the ensemble, forecast, and true values for the 99^{th} percentile, mean of the top percentile, and maximum value for the RNA experiments.

### A.8 Quantifying Distribution Shift

To quantify each forecasting problem, we computed metrics of covariate shift and label shift. Covariate shift measures the change in distribution in covariate space (our sequences and covariates associated with those sequences), while label shift measures a change in the conditional distribution of the outcome given those covariates. For this preliminary analysis, we restricted our study to using model ensemble scores as the main covariate of interest. It would also be reasonable to apply this to edit distances, or higher-dimensional covariates.

TABLE 1
Relative experimental difficulty due to model score-
based covariate and label shift, as measured by the
K-S score between distributions of training and test
ensemble means, and between measurement distributions
from among top-scoring variants, respectively.

| | Model | Model |
|---|---|---|
| | score | score-based |
| | covariate | label |
| Experiment | shift | shift |
| AAV | 0.332 | 0.159 |
| GB1 | 0.960 | 0.916 |
| GFP | 0.888 | 0.541 |
| Landscape 1 Start 1 | 0.176 | 0.283 |
| Landscape 1 Start 2 | 0.162 | 0.227 |
| Landscape 1 Start 3 | 0.329 | 0.518 |
| Landscape 1 Start 4 | 0.579 | 0.575 |
| Landscape 1 Start 5 | 0.214 | 0.521 |
| Landscape 2 Start 1 | 0.371 | 0.606 |
| Landscape 2 Start 2 | 0.402 | 0.094 |
| Landscape 2 Start 3 | 0.283 | 0.830 |
| Landscape 2 Start 4 | 0.285 | 0.794 |
| Landscape 2 Start 5 | 0.381 | 0.747 |

To measure model score covariate shift, we can apply a 2-sample Kolmogorov-Smirnov test to the entire distributions of model scores for S⁰ and S¹. This gives us a measure on a common scale from no shift (0) to completely disjoint supports (1).

Measuring model score-based label shift precisely is challenging in our setting, since our data regularly violates the common assumption for label shift research that the test set output support is a subset of the training set support, so we cannot calculate ratios between the density functions. Instead, we again us the 2-sample K-S test, this time comparing distributions of Y⁰ and Y¹ but conditioned on high model scores (defined as the 90th percentile of the training set distribution and above).

These metrics are showed in Table 1. We note that the AAV experiment, where the forecast performed especially well, had a lesser degree of covariate and label shift compared to other experiments. At the other extreme, the GB1 experiment had extreme covariate and label shift, and while the forecasting method improved upon the ensemble prediction directionally, the forecast produced very low confidence interval coverage for this experiment. This suggests a possible connection between shift scores and forecasting difficulty. On the other hand, looking at the RNA experiments and consider one landscape at a time allows us to potentially isolate the relationship between these covariate shift metrics and forecasting performance.

### A.9 Example Forecasting Algorithm

See Algorithm 1 for a complete description of the forecasting algorithm described in Section A.3 (excluding the extensions in Section A.3.4).

```
  Algorithm 1 Inferring Gaussian mixture model parameters from a set of normally
distributed model scores
  Input: a training set (S⁰, Y⁰) and test set (S¹) with model values mᵢⱼ for each sᵢ ∈ S^{O} ∪
S¹.
  Returns: (pᵢ, μᵢ⁺, μᵢ⁻, σᵢ⁺, σᵢ⁻) for each i ∈ S¹
  Learn cutoff value y_{mid} from Y⁰ (e.g., by using the method described in Nobuyuki Otsu,
“A threshold selection method from gray-level histograms”, IEEE transactions on systems,
man, and cybernetics, 9(1): 62-66, 1979)
  for sᵢ in S⁰ do:
  $Compute {μ}_{i} = \frac{{Σ}_{j} {m}_{i j}}{J} ( model ensemble means )$
  $Compute {σ}_{i}^{2} = \frac{{Σ}_{j} {( {m}_{ij} - {μ}_{i} )}^{2}}{J} ( model ensemble variance )$
  Compute resᵢ² = (yᵢ − μᵢ)² (squared residuals of model ensemble means)
  Compute Iᵢ⁺ = 1 if Yᵢ⁰ < y_{mid} and 0 otherwise
  end for
  Define S⁰⁺ = {i|Yᵢ⁰ > y_{mid}} (training subset for “functional” sequences”
  Define S⁰⁻ = {i|Yᵢ⁰ < y_{mid}} (training subset for “broken” sequences″
  Train isotonic model IRₚ on pairs (μᵢ, Iᵢ⁺ ) for i ∈ S⁰
  Train isotonic model IR_{μ}_{⁺} on pairs (μᵢ, yᵢ) for i ∈ S⁰⁺
  Train isotonic model IR_{μ}_{^{−}} on pairs (μᵢ, yi) for i ∈ S⁰⁻
  Train isotonic model IR_{σ}_{⁺} on pairs (σᵢ², resᵢ² ) for i ∈ S⁰⁺
  Train isotonic model IR_{σ}_{^{−}} on pairs (σᵢ², resᵢ²) for i ∈ S⁰⁻
  for sᵢ in S¹ do:
  $Compute {μ}_{i} = \frac{{Σ}_{j} {m}_{i j}}{J} ( model ensemble means )$
  $Compute {σ}_{i}^{2} = \frac{{Σ}_{j} {( {m}_{ij} - {μ}_{i} )}^{2}}{J} ( model ensemble variance )$
  Compute (pᵢ, μᵢ⁺, μᵢ⁻, σᵢ⁺, σᵢ⁻) = (IRₚ (μᵢ), I [symbol]  (μᵢ), [symbol]  (μᵢ), [symbol]  (σᵢ), I [symbol]  (σᵢ)
  end for
  return {(pᵢ, μᵢ⁺, μᵢ⁻, σᵢ⁺, σᵢ⁻}
```

Further details related to the techniques described in Section A are described in L. Wheelock, S. Malina, J. Gerold, S. Sinai, “Forecasting labels under distribution-shift for machine-guided sequence design”, arXiv:2211.10422, 2022, which is incorporated herein by reference in its entirety.

The terms “program” or “software” are used herein in a generic sense to refer to any type of computer code or set of processor-executable instructions that can be employed to program a computer or other processor to implement various aspects of embodiments as discussed above. Additionally, it should be appreciated that according to one aspect, one or more computer programs that when executed perform methods of the disclosure provided herein need not reside on a single computer or processor, but may be distributed in a modular fashion among different computers or processors to implement various aspects of the disclosure provided herein.

Processor-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in one or more non-transitory computer-readable storage media in any suitable form. For simplicity of illustration, data structures may be shown to have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements.

Also, various inventive concepts may be embodied as one or more processes, of which examples have been provided. The acts performed as part of each process may be ordered in any suitable way. Accordingly, embodiments may be constructed in which acts are performed in an order different than illustrated, which may include performing some acts simultaneously, even though shown as sequential acts in illustrative embodiments.

The described embodiments can be implemented in various combinations, including the below configurations:

(1) A method for predicting performance of biological sequences, comprising: using at least one computer hardware processor to perform: accessing a plurality of biological sequences and model scores associated with the plurality of biological sequences, wherein the plurality of biological sequences and model scores are generated using a machine learning model trained on training data comprising biological sequences and labels for an attribute of the biological sequences; accessing a statistical model configured to generate output indicating predictions for the attribute of the plurality of biological sequence, wherein the statistical model is configured to allow for at least some of the predictions to occur outside a distribution of labels in the training data; and generating, using the statistical model, the plurality of biological sequences, and the model scores, an output indicating a predicted distribution of labels for the attribute of the plurality of biological sequences.

(2) The method of (1), wherein the statistical model allows for at least some of the predictions to occur outside a range of the distribution of labels in the training data.

(3) The method of (1) or (2), wherein the statistical model is configured to allow for at least some of the predictions to occur outside a distribution of models scores generated by ensembling the model scores.

(4) The method of any of (1)-(3), further comprising determining, using the output indicating the predicted distribution of labels, a likelihood of the plurality of biological sequences comprising at least one biological sequence having a measurement for the attribute greater than the labels.

(5) The method of any of (1)-(4), further comprising determining, using the output indicating the predicted distribution of labels, a number of biological sequences from among the plurality of biological sequences as having a value for the attribute above a threshold value.

(6) The method of any of (1)-(5), wherein the plurality of biological sequences is a first plurality of biological sequences, the method further comprising generating, based on the output indicating the predicted distribution of labels, a second plurality of biological sequences at least in part by using the machine learning model to obtain as output the second plurality of biological sequences.

(7) The method of any of (1)-(6), wherein the predicted distribution of labels of the attribute comprises a distribution of values corresponding to predictions of the attribute for the plurality of biological sequences.

(8) The method of any of (1)-(7), further comprising manufacturing at least some of the plurality of biological sequences.

(9) The method of any of (1)-(8), further comprising: selecting, based on the predicted distribution of labels for the attribute, a subset of the plurality of biological sequences; and manufacturing the subset of the plurality of biological sequences.

(10) The method of any of (1)-(9), wherein the plurality of biological sequences is a first plurality of biological sequences, the model scores is a first set of model scores, and the output is a first output, and wherein the method further comprises: accessing a second plurality of biological sequences and a second set of model scores associated with the second plurality of biological sequences; generating, using the statistical model, the second plurality of biological sequences, and the second set of model scores, a second output indicating a predicted distribution of labels for the attribute for the second plurality of biological sequences; and selecting the first plurality of biological sequences or the second plurality of biological sequences based on the first output and the second output.

(11) The method of any of (10) further comprising: manufacturing, based on the selecting, the first plurality of biological sequences or the second plurality of biological sequences.

(12) The method of any of (1)-(11), wherein the model scores include at least one model score associated with each of the plurality of biological sequences.

(13) The method of any of (1)-(12), wherein the machine learning model includes a regression model, and the model scores include regression estimates associated with the plurality of biological sequences.

(14) The method of any of (1)-(13), wherein generating the output using the statistical model, the plurality of biological sequences, and the model scores further comprises identifying, using the model scores, an estimate for at least one parameter of a probability distribution for the plurality of biological sequences.

(15) The method of any of (1)-(14), wherein generating the output using the statistical model, the plurality of biological sequences, and the model scores further comprises determining, for each of the plurality of biological sequences, a probability distribution.

(16) The method of (15), wherein determining the probability distribution for each of the plurality of biological sequences further comprises identifying estimates for parameters of the probability distribution for each of the plurality of biological sequences based on the model scores.

(17) The method of (16), wherein identifying parameters of the probability distribution for each of the plurality of biological sequences further comprises identifying means and variances for the model scores, each mean and each variance corresponding to one biological sequence of the plurality of biological sequences.

(18) The method of any of (15)-(17), wherein determining the probability for each of the plurality of biological sequences further comprises determining a posterior distribution for each of the plurality of biological sequences and identifying estimates for parameters of the posterior distribution for each of the plurality of biological sequences based on the model scores.

(19) The method of any of (15)-(18), wherein the statistical model comprises a multimodal model having a first mode and a second mode, and identifying estimates for parameters of the probability distribution for each of the plurality of biological sequences further comprises identifying a first set estimates for parameters associated with the first mode and a second set of estimates for parameters associated with the second mode.

(20) The method of any of (19), wherein the statistical model includes at least one Gaussian mixture model comprising the first mode and the second mode.

(21) The method of any of (19) or (20), wherein the statistical model includes a first regression model trained on biological sequences and labels associated with the first mode and a second regression model trained on biological sequences and labels associated with the second mode, and wherein identifying estimates for parameters of the probability distribution further comprises using the first regression model to identify the first set of estimates for parameters associated with the first mode and using the second regression model to identify the second set of estimates for parameters associated with the second mode.

(22) The method of any of (19)-(21), wherein generating the output indicating the predicted distribution of labels for the attribute of the plurality of biological sequences further comprises using the first set of estimates for parameters associated with the first mode to generate a predicted distribution of labels associated with the first mode and using the second set of estimates for parameters associated with the second mode to generate a predicted distribution of labels associated with the second mode.

(23) The method of any of (1)-(22), wherein the statistical model includes a parameter relating to a sequence distance metric, and generating the output indicating the predicted distribution of labels further comprises using an estimate for the parameter relating to a sequence distance metric to adjust the predictions generated by the statistical model.

(24) The method of any of (1)-(23), wherein the plurality of biological sequences comprises polypeptide sequences.

(25) The method of any of (1)-(24), wherein the plurality of biological sequences comprises sequences for dependoparvovirus capsid proteins.

(26) The method of any of (25), wherein the plurality of biological sequences comprises variants of a wild-type dependoparvovirus capsid protein.

(27) The method of (25) or (26), wherein the attribute is transduction efficiency for a target tissue type, and the labels comprise values of transduction efficiency for dependoparvovirus capsid proteins.

(28) The method of (25) or (26), wherein the attribute includes packaging efficiency, and the labels comprise values of packaging efficiency for the dependoparvovirus capsid proteins.

(29) A system comprising: at least one hardware processor; and at least one non-transitory computer-readable storage medium storing processor-executable instructions that, when executed by the at least one hardware processor, cause the at least one hardware processor to perform the method comprising: accessing a plurality of biological sequences and model scores associated with the plurality of biological sequences, wherein the plurality of biological sequences and model scores are generated using a machine learning model trained on training data comprising biological sequences and labels for an attribute of the biological sequences; accessing a statistical model configured to generate output indicating predictions for the attribute of the plurality of biological sequence, wherein the statistical model is configured to allow for at least some of the predictions to occur outside a distribution of labels in the training data; and generating, using the statistical model, the plurality of biological sequences, and the model scores, an output indicating a predicted distribution of labels for the attribute of the plurality of biological sequences.

(30) At least one non-transitory computer-readable storage medium storing processor-executable instructions that, when executed by at least one hardware processor, cause the at least one hardware processor to perform the method comprising: accessing a plurality of biological sequences and model scores associated with the plurality of biological sequences, wherein the plurality of biological sequences and model scores are generated using a machine learning model trained on training data comprising biological sequences and labels for an attribute of the biological sequences; accessing a statistical model configured to generate output indicating predictions for the attribute of the plurality of biological sequence, wherein the statistical model is configured to allow for at least some of the predictions to occur outside a distribution of labels in the training data; and generating, using the statistical model, the plurality of biological sequences, and the model scores, an output indicating a predicted distribution of labels for the attribute of the plurality of biological sequences.

All definitions, as defined and used herein, should be understood to control over dictionary definitions, and/or ordinary meanings of the defined terms.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the techniques described herein in detail, various modifications, and improvements will readily occur to those skilled in the art. Such modifications and improvements are intended to be within the spirit and scope of the disclosure. Accordingly, the foregoing description is by way of example only, and is not intended as limiting. The techniques are limited only as defined by the following claims and the equivalents thereto.

What is claimed is:

1. A method for predicting performance of biological sequences, comprising:
using at least one computer hardware processor to perform:
accessing a plurality of biological sequences and model scores associated with the plurality of biological sequences, wherein the plurality of biological sequences and model scores are generated using a machine learning model trained on training data comprising biological sequences and labels for an attribute of the biological sequences;
accessing a statistical model configured to generate output indicating predictions for the attribute of the plurality of biological sequence, wherein the statistical model is configured to allow for at least some of the predictions to occur outside a distribution of labels in the training data; and
generating, using the statistical model, the plurality of biological sequences, and the model scores, an output indicating a predicted distribution of labels for the attribute of the plurality of biological sequences.

2. The method of claim 1, wherein the statistical model allows for at least some of the predictions to occur outside a range of the distribution of labels in the training data.

3. The method of claim 1 or any other preceding claim, wherein the statistical model is configured to allow for at least some of the predictions to occur outside a distribution of models scores generated by ensembling the model scores.

4. The method of claim 1 or any other preceding claim, further comprising determining, using the output indicating the predicted distribution of labels, a likelihood of the plurality of biological sequences comprising at least one biological sequence having a measurement for the attribute greater than the labels.

5. The method of claim 1 or any other preceding claim, further comprising determining, using the output indicating the predicted distribution of labels, a number of biological sequences from among the plurality of biological sequences as having a value for the attribute above a threshold value.

6. The method of claim 1 or any other preceding claim, wherein the plurality of biological sequences is a first plurality of biological sequences, the method further comprising generating, based on the output indicating the predicted distribution of labels, a second plurality of biological sequences at least in part by using the machine learning model to obtain as output the second plurality of biological sequences.

7. The method of claim 1, wherein the predicted distribution of labels of the attribute comprises a distribution of values corresponding to predictions of the attribute for the plurality of biological sequences.

8. The method of claim 1 or any other preceding claim, further comprising manufacturing at least some of the plurality of biological sequences.

9. The method of claim 1 or any other preceding claim, further comprising:
selecting, based on the predicted distribution of labels for the attribute, a subset of the plurality of biological sequences; and
manufacturing the subset of the plurality of biological sequences.

10. The method of claim 1 or any other preceding claim, wherein the plurality of biological sequences is a first plurality of biological sequences, the model scores is a first set of model scores, and the output is a first output, and wherein the method further comprises:
accessing a second plurality of biological sequences and a second set of model scores associated with the second plurality of biological sequences;
generating, using the statistical model, the second plurality of biological sequences, and the second set of model scores, a second output indicating a predicted distribution of labels for the attribute for the second plurality of biological sequences; and
selecting the first plurality of biological sequences or the second plurality of biological sequences based on the first output and the second output.

11. The method of claim 10 or any other preceding claim, further comprising:
manufacturing, based on the selecting, the first plurality of biological sequences or the second plurality of biological sequences.

12. The method of claim 1 or any other preceding claim, wherein the model scores include at least one model score associated with each of the plurality of biological sequences.

13. The method of claim 1 or any other preceding claim, wherein the machine learning model includes a regression model, and the model scores include regression estimates associated with the plurality of biological sequences.

14. The method of claim 1 or any other preceding claim, wherein generating the output using the statistical model, the plurality of biological sequences, and the model scores further comprises identifying, using the model scores, an estimate for at least one parameter of a probability distribution for the plurality of biological sequences.

15. The method of claim 1 or any other preceding claim, wherein generating the output using the statistical model, the plurality of biological sequences, and the model scores further comprises determining, for each of the plurality of biological sequences, a probability distribution.

16. The method of claim 15 or any other preceding claim, wherein determining the probability distribution for each of the plurality of biological sequences further comprises identifying estimates for parameters of the probability distribution for each of the plurality of biological sequences based on the model scores.

17. The method of claim 16 or any other preceding claim, wherein identifying parameters of the probability distribution for each of the plurality of biological sequences further comprises identifying means and variances for the model scores, each mean and each variance corresponding to one biological sequence of the plurality of biological sequences.

18. The method of claim 15 or any other preceding claim, wherein determining the probability for each of the plurality of biological sequences further comprises determining a posterior distribution for each of the plurality of biological sequences and identifying estimates for parameters of the posterior distribution for each of the plurality of biological sequences based on the model scores.

19. The method of claim 15 or any other preceding claim, wherein the statistical model comprises a multimodal model having a first mode and a second mode, and identifying estimates for parameters of the probability distribution for each of the plurality of biological sequences further comprises identifying a first set estimates for parameters associated with the first mode and a second set of estimates for parameters associated with the second mode.

20. The method of claim 19 or any other preceding claim, wherein the statistical model includes at least one Gaussian mixture model comprising the first mode and the second mode.

21. The method of claim 19 or any other preceding claim, wherein the statistical model includes a first regression model trained on biological sequences and labels associated with the first mode and a second regression model trained on biological sequences and labels associated with the second mode, and wherein identifying estimates for parameters of the probability distribution further comprises using the first regression model to identify the first set of estimates for parameters associated with the first mode and using the second regression model to identify the second set of estimates for parameters associated with the second mode.

22. The method of claim 19, wherein generating the output indicating the predicted distribution of labels for the attribute of the plurality of biological sequences further comprises using the first set of estimates for parameters associated with the first mode to generate a predicted distribution of labels associated with the first mode and using the second set of estimates for parameters associated with the second mode to generate a predicted distribution of labels associated with the second mode.

23. The method of claim 1 or any other preceding claim, wherein the statistical model includes a parameter relating to a sequence distance metric, and generating the output indicating the predicted distribution of labels further comprises using an estimate for the parameter relating to a sequence distance metric to adjust the predictions generated by the statistical model.

24. The method of claim 1 or any other preceding claim, wherein the plurality of biological sequences comprises polypeptide sequences.

25. The method of claim 1 or any other preceding claim, wherein the plurality of biological sequences comprises sequences for dependoparvovirus capsid proteins.

26. The method of claim 25 or any other preceding claim, wherein the plurality of biological sequences comprises variants of a wild-type dependoparvovirus capsid protein.

27. The method of claim 25 or any other preceding claim, wherein the attribute is transduction efficiency for a target tissue type, and the labels comprise values of transduction efficiency for dependoparvovirus capsid proteins.

28. The method of claim 25 or any other preceding claim, wherein the attribute includes packaging efficiency, and the labels comprise values of packaging efficiency for the dependoparvovirus capsid proteins.

29. A system comprising:
at least one hardware processor; and
at least one non-transitory computer-readable storage medium storing processor-executable instructions that, when executed by the at least one hardware processor, cause the at least one hardware processor to perform the method of any one of claims 1-28.

30. At least one non-transitory computer-readable storage medium storing processor-executable instructions that, when executed by at least one hardware processor, cause the at least one hardware processor to perform the method of any one of claims 1-28.
